(19) United States
(12) Patent Application Publication
(10) Pub. No.: US 2026/0329917 A1
(43) Pub. Date: Oct. 22, 2026

(54) DETERMINING REPAIR INFORMATION VIA AUTOMATED ANALYSIS OF STRUCTURED AND UNSTRUCTURED REPAIR DATA

(71) Applicant: THE COLLECTIVE JOURNEY, LLC, Herber City, UT (US)

(72) Inventors: Rami Hashish, Santa Monica, CA (US); Vladyslav Borysenko, Kyiv (UA)

(73) Assignee: THE COLLECTIVE JOURNEY, LLC, Herber City, UT (US)

(21) Appl. No.: 19/659,301

(22) Filed: Apr. 27, 2026

Related U.S. Application Data
(63) Continuation of application No. 18/131,278, filed on Apr. 5, 2023, now Pat. No. 12,632,483.
(60) Provisional application No. 63/331,414, filed on Apr. 15, 2022.

(51) Int. Cl. G06F 16/338; G06F 16/34; G06F 40/40; G06V 30/413

(52) U.S. Cl. CPC G06F 16/338; G06F 16/345; G06F 40/40; G06V 30/413

(57) ABSTRACT

Techniques are described for determining data of multiple types for an indicated entity from documents describing activities performed by multiple service providers, using both structured and unstructured data formats. Using a combination of image-based and text-based analysis, content groupings are determined within the documents, including multiple content groupings within a document. Sections are determined within the content groupings by identifying structural elements, combining some to form sections while selecting others as separate sections. Data is extracted from the sections and linked across multiple documents to form an aggregated summary for the indicated entity, including values for attributes of the entity. The aggregated summary may be presented via a graphical user interface (GUI). Queries received via the GUI may be answered with response information including the attribute values.

### CROSS REFERENCE TO RELATED APPLICATIONS

This application is a continuation of, and claims priority to, and the benefit of, U.S. patent application Ser. No. 18/131,278, filed Apr. 5, 2023 and entitled “Determining Repair Information Via Automated Analysis Of Structured And Unstructured Repair Data,” which claims priority to, and the benefit of, U.S. Provisional Patent Application No. 63/331,414, filed Apr. 15, 2022 and entitled “Determining Repair Information Via Automated Analysis Of Structured And Unstructured Repair Data,” which are hereby incorporated by reference in their entirety.

### TECHNICAL FIELD

The following disclosure relates generally to automated techniques for determining repair information based at least in part on analysis of structured and unstructured repair data, such as to perform a combination of automated image-based and textual-based analysis of the repair data for use in automatically determining repairs of specific types that have been made to computing devices or for other types of repair and maintenance information.

### BACKGROUND

An abundance of information is available to users on a wide variety of topics from a variety of sources. For example, portions of the World Wide Web (“the Web”) are akin to an electronic library of documents and other data resources distributed over the Internet, with billions of documents available, including groups of documents directed to various specific topic areas. In addition, various other information is available via other communication mediums.

However, existing search engines and other techniques for identifying information of interest suffer from various problems. Non-exclusive examples include a difficulty in identifying and using specific search terms in a useful manner, difficulty in finding answers specific to a particular topic of interest, receiving an overabundance of responses to a query that are too extensive to easily review and with many or most (or sometimes all) being only partially relevant or not relevant to the query (and that thus obscure relevant information if it actually is included in the responses), etc.

### BRIEF DESCRIPTION OF THE DRAWINGS

The subject matter of the present disclosure is particularly pointed out and distinctly claimed in the concluding portion of the specification. A more complete understanding of the present disclosure, however, may be obtained by referring to the detailed description and claims when considered in connection with the drawing figures. Elements with like element numbering through the figures are intended to be the same or similar.

FIGS. 1A and 1B are network diagrams illustrating an example environment and a system for performing described techniques, including automatically determining repair or maintenance information via automated analysis of structured and unstructured data, such as for use in responding to queries related to computing devices or to other types of repair or maintenance information.

FIGS. 2A-2W illustrate examples of performing described techniques, including automatically determining repair or maintenance information for computing devices or other types of repair or maintenance information, and using the determined information in one or more manners.

FIG. 3 is a block diagram illustrating an example of computing systems for use in performing described techniques, including automatically determining repair or maintenance information for computing devices or other types of repair or maintenance information.

FIG. 4 illustrates a flow diagram of an example embodiment of an Automated Repair Information Determination (“ARID”) system routine.

FIG. 5 illustrates a flow diagram of an example embodiment of an ARID Repair Information Analyzer component routine.

FIG. 6 illustrates a flow diagram of an example embodiment of an ARID Query Matching and Response Determination component routine.

FIG. 7 illustrates a flow diagram of an example embodiment of a client device routine.

### DETAILED DESCRIPTION

The present disclosure describes techniques for using computing devices to perform automated operations related to identifying and using repair and/or maintenance information-such techniques may include, for example, extracting and linking data about repair and maintenance activities performed on various devices (e.g., multiple devices of multiple types), determining specific repair and/or maintenance information of one or more specified types in response to queries (e.g., for one or more particular such devices that are identified based on those queries), and subsequently using the identified repair and/or maintenance information in one or more further automated manners in some situations. In at least some embodiments, the identified information about repair and/or maintenance activities relates to computing devices (e.g., computer systems, such as desktop computers, laptop computers, tablet computers, server computing systems, etc. ; smart phones; etc.), with the identified repair and/or maintenance activity information (referred to subsequently herein at times as “repair” activities or “repair” information but intended to also include maintenance activities or information, respectively, unless otherwise indicated explicitly or by context) being analyzed in some such embodiments and situations to identify areas for corrective activities (e.g., additional repair activities to be performed, problems in previous repair activities to be corrected, etc.) for one or more target computing devices, and the use of such information may include automatically initiating some or all such corrective activities on the target computing device(s). In addition, in at least some embodiments, various techniques may be used to improve speed and/or accuracy of determined responses to received queries, including analyzing and validating data about repair activities for a plurality of devices (e.g., identifying particular repair and/or maintenance events and/or associated objects used, determining associated timelines, etc.), identifying and removing duplicative data (e.g., repair data records), and then identifying subsets of such data matching received queries. Additional details are included below regarding the automated analysis and use of repair information, and some or all of the techniques described herein may, in at least some embodiments, be performed via automated operations of an Automated Repair Information Determination (“ARID”) system, as discussed further below.

In at least some embodiments, the described techniques include analyzing repair information for a number of types of repair activities and linking extracted repair data for particular entities, such as from multiple source documents (from one or more repair activity providers) and/or across multiple repair encounters, and using a combination of both image-based and text-based analyses. For example, a group of information specific to one or more types of repair activities (e.g., involving some or all repair activities for a specific type or class of computing device or for a specific computing device) may be identified (e.g., in a plurality of documents from a plurality of sources and including both structured data and unstructured data, and with information for a particular device or other entity potentially identified in multiple documents from multiple sources and linked together), and may be analyzed to separate that group of information into smaller groupings of related data (e.g., per device or other entity, per repair activity encounter, per repair activity type, etc.). In at least some embodiments, the analysis activities for a data grouping may include using a language model specific to a type of repair activity (e.g., to extract relevant data from different groups of related text and other data, such as based at least in part on categorization activities performed for the groups of related text and other data). Furthermore, in some embodiments, the described techniques include automated operations of the ARID system to anonymize or otherwise redact some of the repair information and/or other associated information about particular devices or other entities.

As noted above, the described techniques may include obtaining documents that include repair data (and optionally additional types of data), and analyzing contents of the documents to extract repair data of one or more types. In at least some embodiments, the analysis of a particular target document with repair data may include using a combination of image-based analysis and text-based analysis to identify one or more groupings of related content (also referred to herein as “content groupings”) in the document, such as to correspond to separate original documents that were combined to form the target document and are now sub-documents within the target document, or to other types of groupings of related content in the target document (e.g., content of different types, content that is generated at different times and/or in different manners, etc.)—in at least some such embodiments, a first trained neural network model performs a pairwise analysis of each pair of pages of the target document (e.g., of images of the pages) in order to initially determine (e.g., predict) whether those two pages are part of the same or different content groupings (i.e., whether or not a content grouping boundary, or ‘split’, exists between two different content groupings to which those two pages correspond, such as a split occurring between the two pages, including to correspond to situations in which the content grouping boundary identifies where one original document in the target document ends and another original document in the target document begins), and a second prediction model (e.g., a trained transformer neural network model or other type of neural network model) then analyzes text of the target document surrounding each determined split point between content groupings to validate (or overrule) the initial content grouping split determination.

Once the separate content groupings are identified in the target document (e.g., separate original documents that were combined to form the target document), each such separate content grouping is then analyzed to categorize that content grouping (e.g., from an enumerated list or set of content grouping categories), including in at least some embodiments to use text-based analysis, such as by a third prediction model (e.g., a trained transformer neural network model or other type of neural network model) that initially classifies the content of that content grouping as belonging to a particular content grouping category based on text of the content grouping (e.g., using only a subset of the content from the content grouping, such as 1-3 pages, and in some cases by using transformer neural network embeddings from the first 3 pages or the first 512 tokens, whichever comes first), and optionally with a subsequent image-based analysis of the content of that content grouping to validate (or overrule) that initial content grouping categorization (e.g., using a fourth trained neural network model). In at least some embodiments, only certain categories of content groupings are then further analyzed to extract relevant repair data, with content groupings of other categories (e.g., bills, cover letters, transmittal documents, etc.) being excluded from further analysis (but still optionally used in other manners).

For the identified content groupings that are remaining after the optional exclusion of some content groupings whose determined content grouping categories meet one or more specified criteria, the described techniques in at least some embodiments further include performing analysis of a particular content grouping having repair data to identify subsets of the content grouping that correspond to different repair-related encounters, such as at different times and/or by different repair providers (e.g., corresponding to different repair or maintenance sessions involving interactions of a repair provider with a particular target device or other target entity) for example, such identification of an encounter may be based at least in part on one or more repair activities that occur within a defined time period (e.g., a defined quantity of one or more minutes or hours or days) and/or are otherwise indicated as being associated together based on the content.

For each such content grouping subset corresponding to an encounter (if identified) or each such content grouping overall (if encounters are not identified), the described techniques may further include performing analysis of the content of that encounter (if available) or of that content grouping (if not) by using a combination of image-based analysis and text-based analysis to identify one or more sections in that content, such as for sections each having one or more structural elements corresponding to separate types of information and/or formatting within that content (e.g., titles, headers, footers, form data, substantive repair data, etc.). For example, in at least some such embodiments, a fifth trained computer vision object-detection model (e.g., Detectron2) analyzes images of that content for the encounter's content grouping subset or overall content grouping (e.g., separate images for each page) in order to determine (e.g., predict) separate structural element objects of that content (e.g., based on formatting and/or location of different subsets of that content), such as to identify titles, sub-titles, tables, ‘forms’ with key-value pairs, questionnaires, header/footers, images, graphs, substantive data (e.g., text, numbers, etc.), page numbers, paragraph and line numbers, etc., and to optionally assign corresponding labels to each structural element. In addition, in at least some such embodiments, text may be extracted and analyzed using one or more trained OCR (optical character recognition) models. Sections may then be identified by, for example, combining related structural elements (e.g., based at least in part on structural element labels, such as to combine a title and/or sub-title with following substantive data), while treating other structural elements (e.g., tables, forms, questionnaires, images, graphs, etc.) as separate sections—in at least some such embodiments, some types of structural elements (e.g., headers, footers, page numbers, etc.) are further excluded from inclusion in any of the sections and from further section-based analysis. Once the separate sections are identified, each such separate section is then analyzed to categorize that section (e.g., from an enumerated list or set of section categories), including in at least some embodiments to use a sixth trained prediction model (e.g., a transformer neural network model or other type of neural network model, such as based on a Bert NLP, or Natural Language Processing, model) that classifies the content of that section as belonging to a particular section category based at least in part on text of that section. In at least some embodiments, only certain categories of sections are then further analyzed to extract relevant repair data, with sections of other categories (e.g., headers, footers, page and line numbering, etc.) being excluded from further analysis (but still optionally used in other manners).

For the identified sections that are remaining after the optional exclusion of some section categories, the described techniques in at least some embodiments further include performing analysis of a particular section having repair data to extract repair data from the section and to identify one or more devices or other entities to which the repair data corresponds, such as to identify particular repair activities performed and corresponding results for particular associated device(s) or other entity(ies), including to determine values for particular attributes of the associated device(s) or other entity(ies). For example, additional text may be extracted and analyzed in at least some embodiments using one or more seventh trained OCR models, such as to handle some specialized types of sections (e.g., tables, forms, graphs, etc.)—as one example, information in some such specialized section types may provide information about particular attributes of an associated device or other entity, such as based on a table column and cell value, a key-value pair of a form, one or more axes and values of a graph, etc. In addition, the analysis of the text may include separating it into different predicted types or topics, with non-exclusive examples including general information (e.g., names, dates, addresses, etc.), diagnosis of problems needing repair, treatments of particular repair problems, care plans for planned further repair activities to manage or solve particular repair problems, a history of past repair activities and problems, review by one repair provider of repair activities by others, etc. The described techniques may further optionally include annotating the other documents and/or content groupings with information determined from the automated analysis, such as to identify encounters and/or sections and/or structural elements with bounding boxes (or other visual indications) and/or to provide associated determined categories and/or topics/types (e.g., to provide corresponding information to human end users and/or to enable review and optional revision by human annotator users). In addition, the described techniques may further include linking extracted data for a particular associated device or other entity from different documents and/or content groupings and/or encounters and/or sections and/or structural elements, including in some embodiments to provide a comprehensive aggregation of all available repair data for that associated device or other entity.

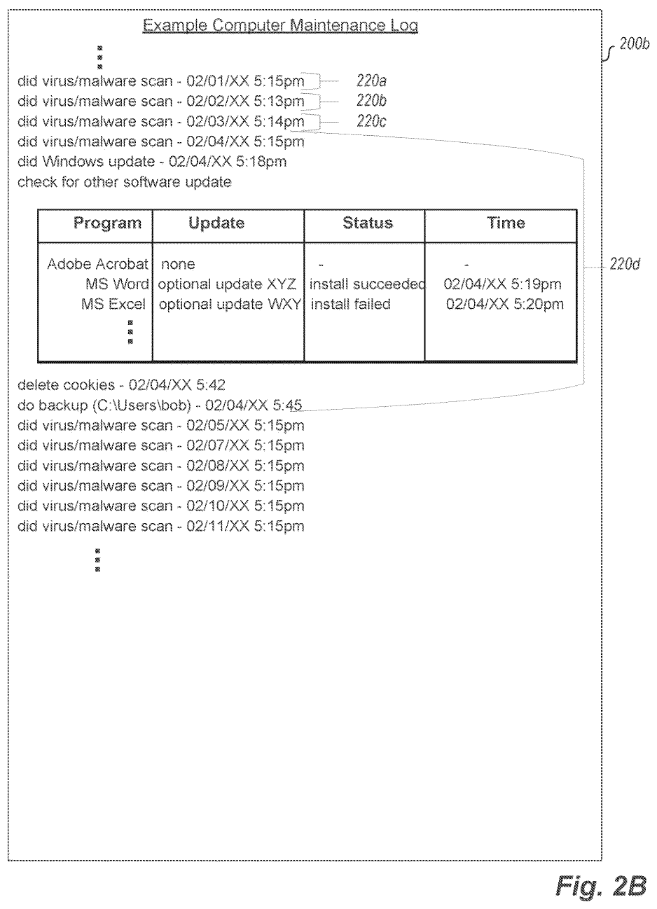

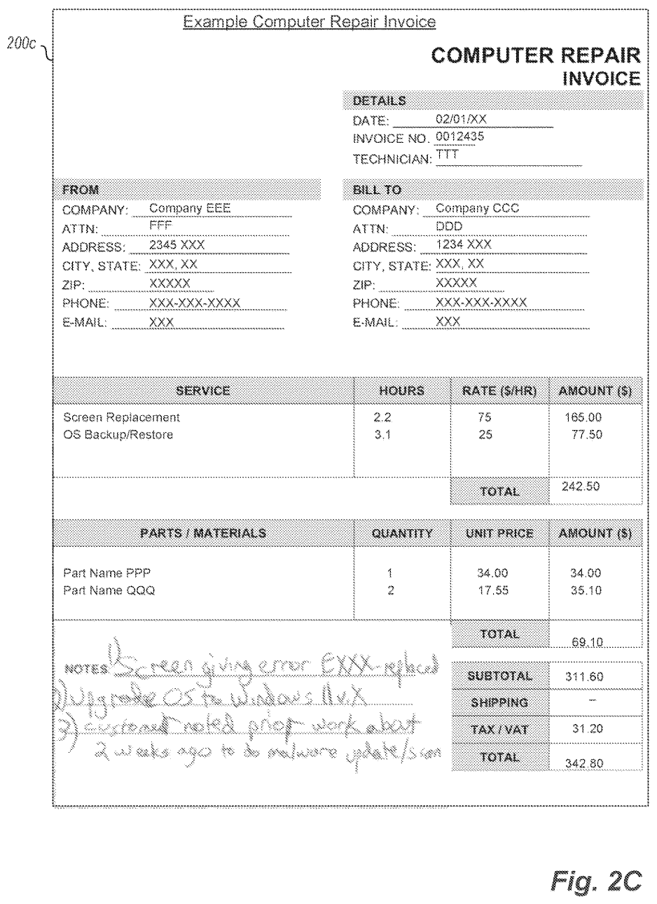

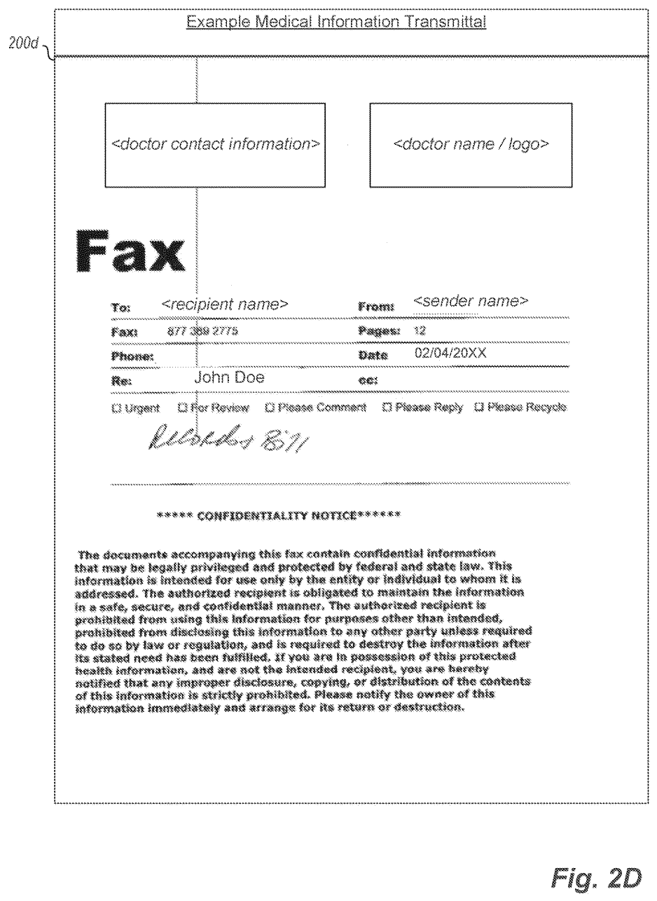

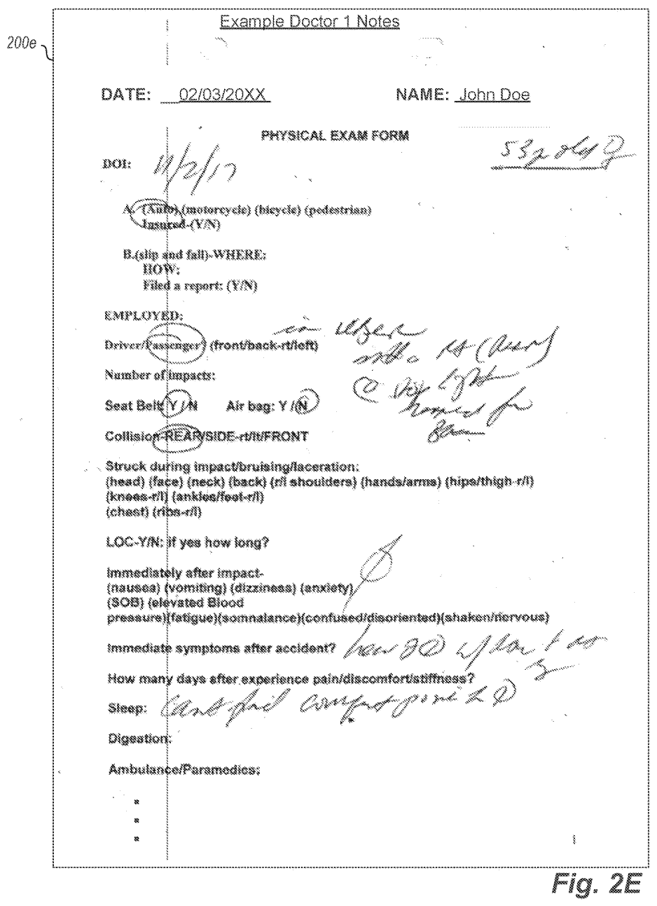

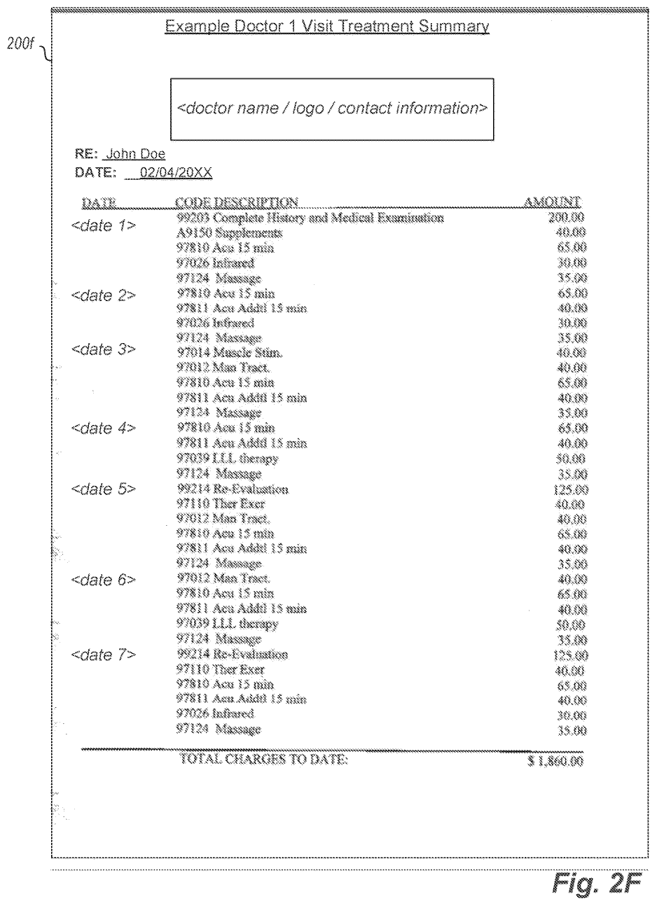

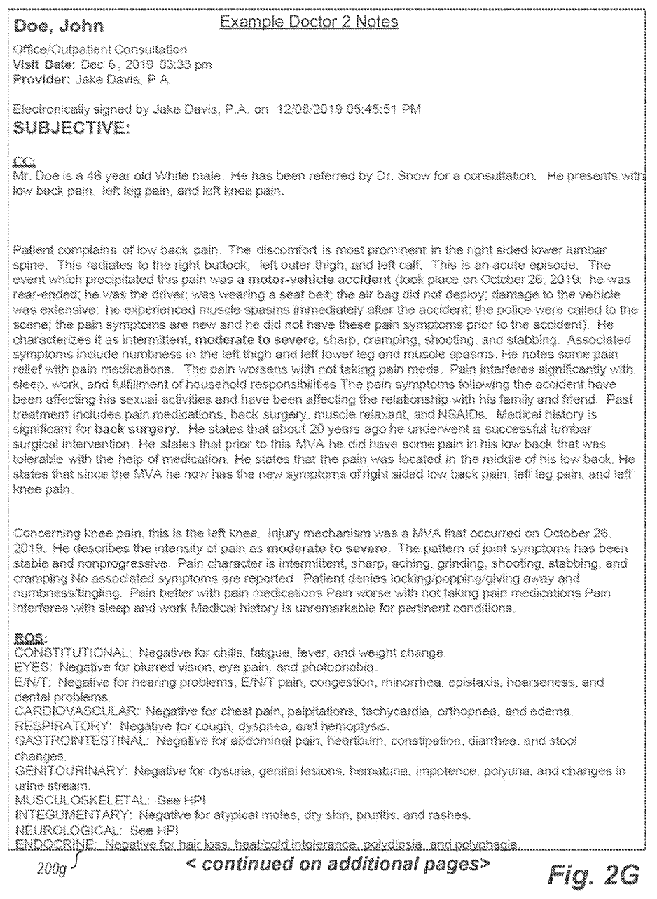

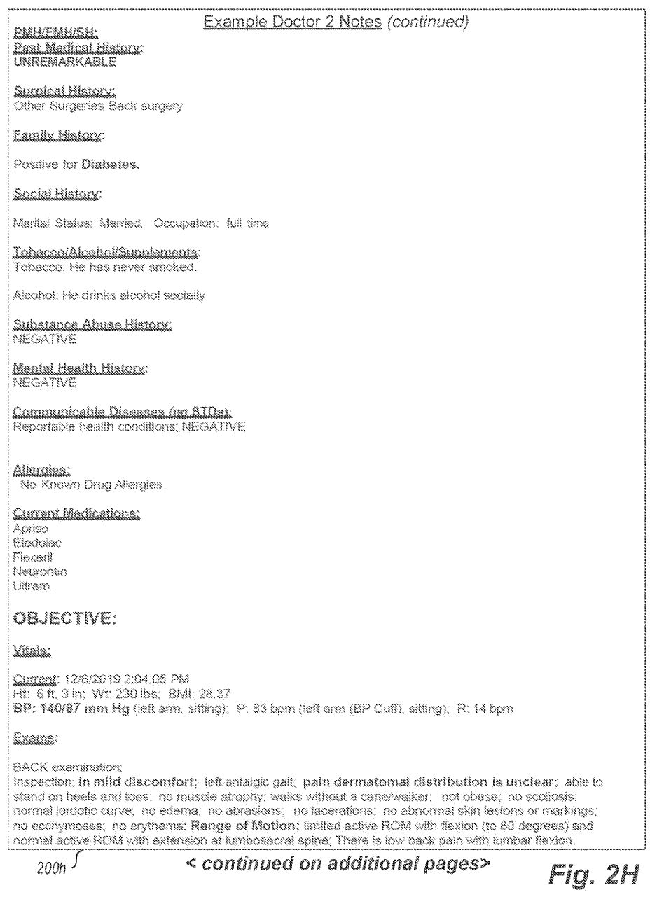

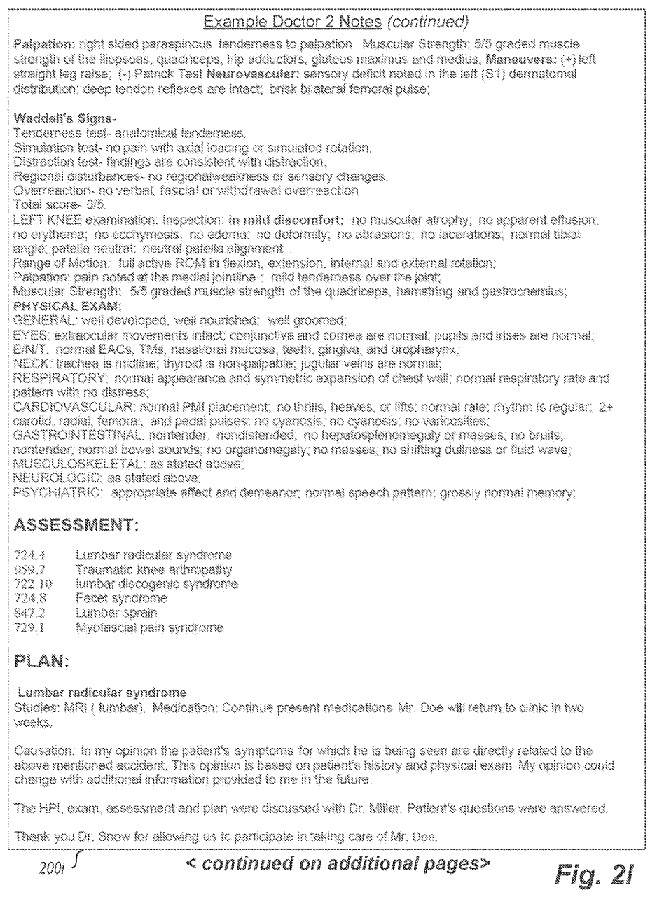

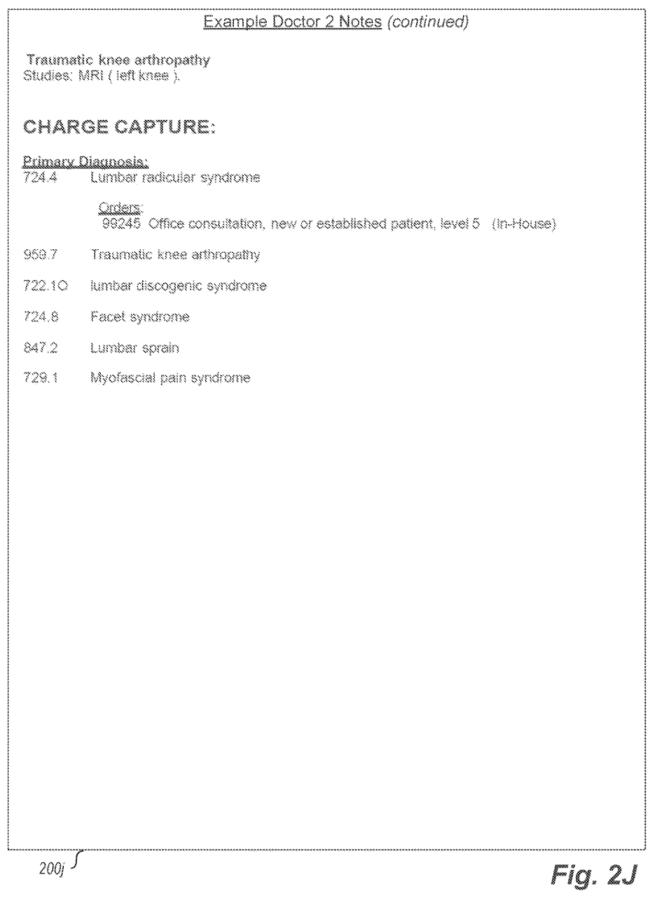

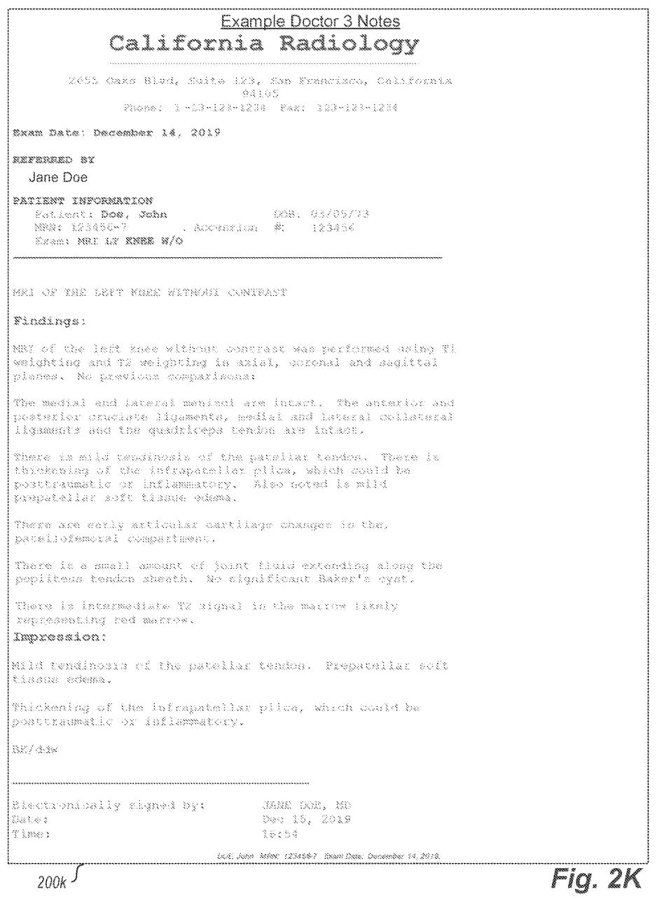

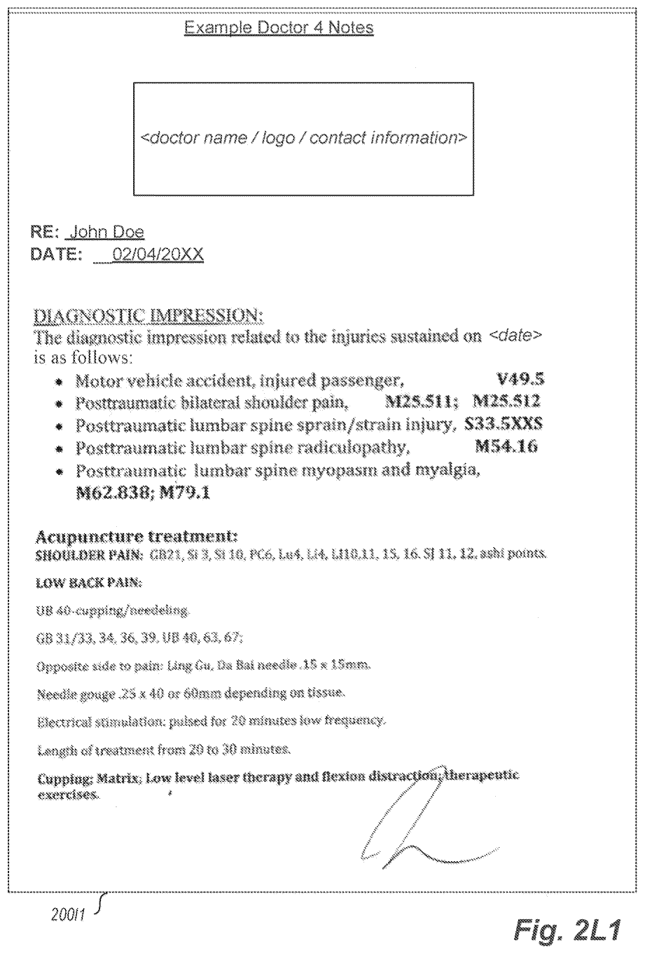

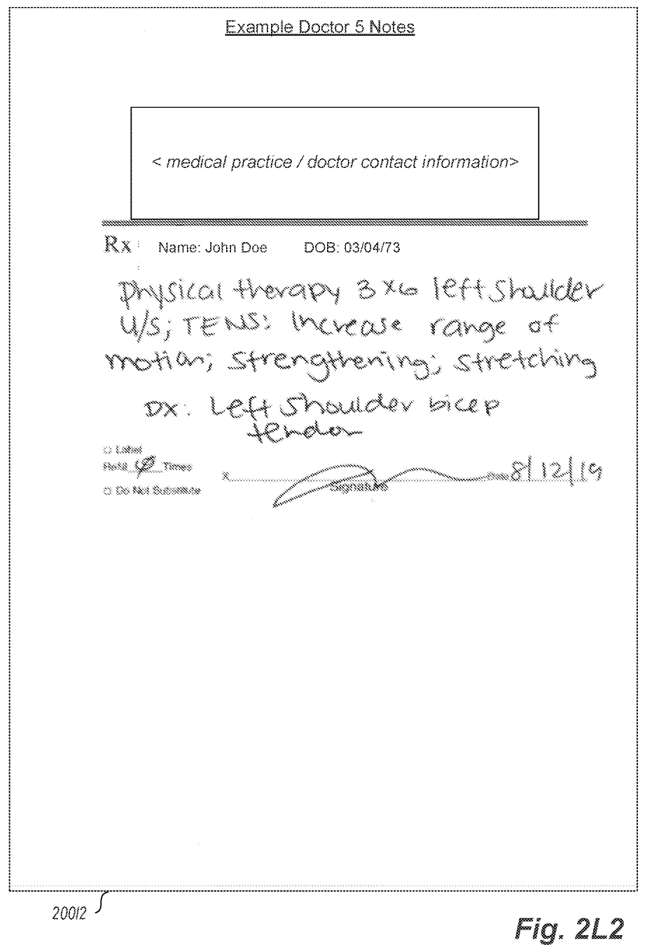

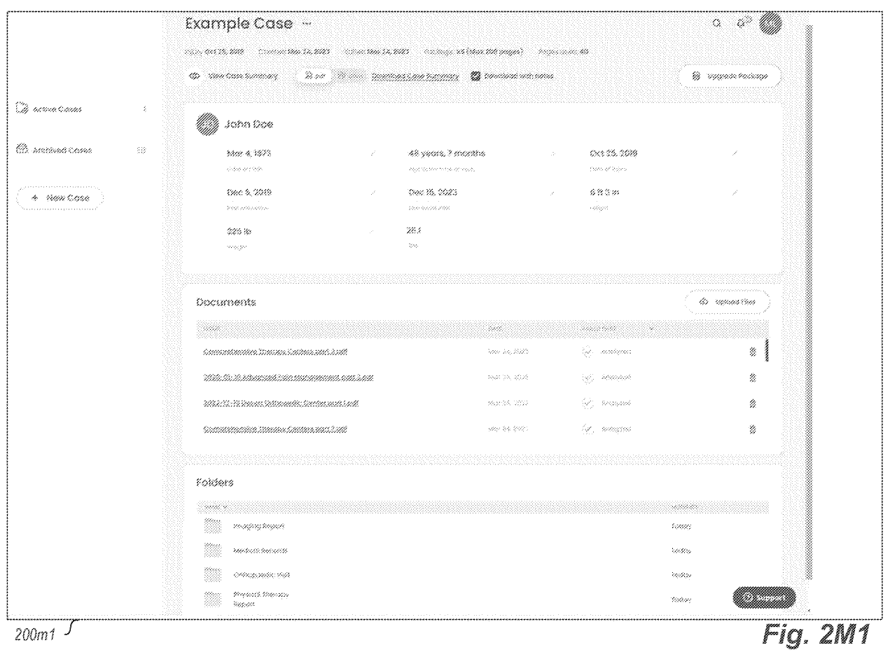

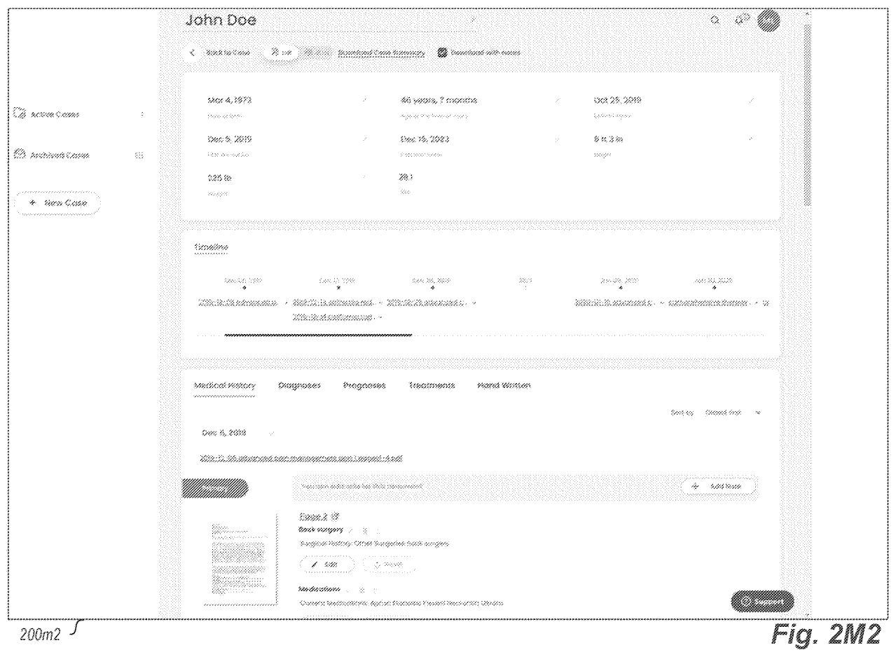

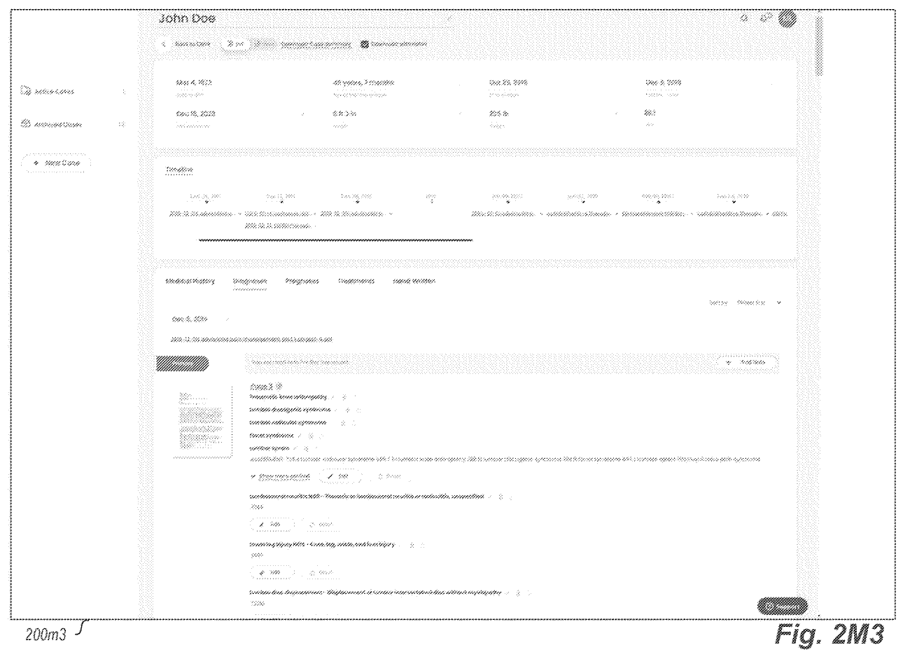

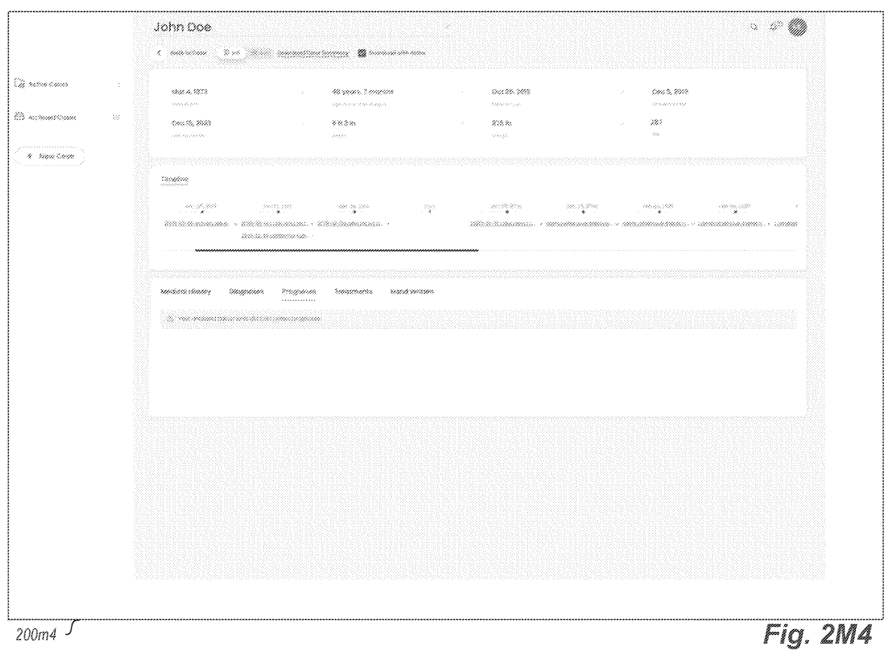

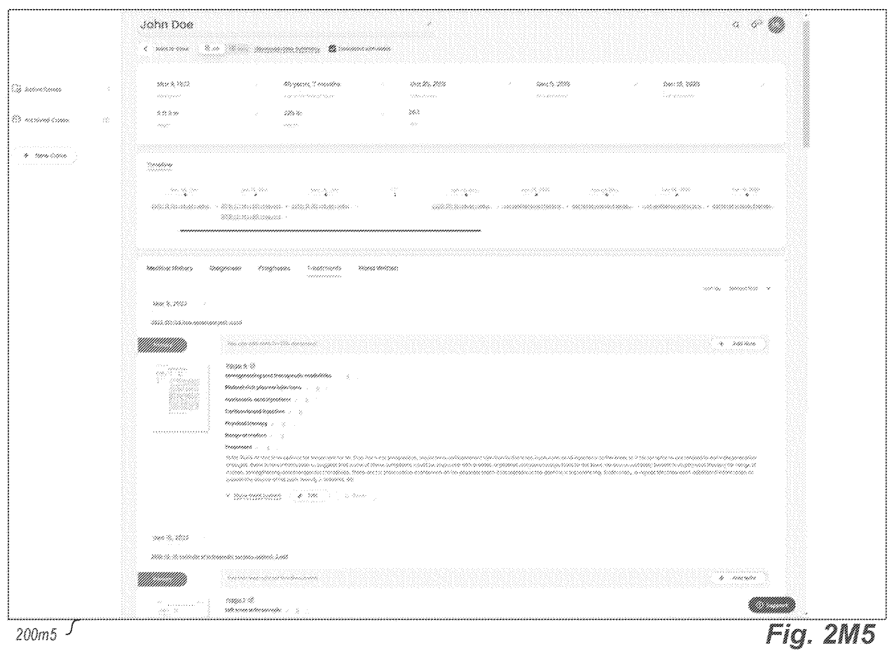

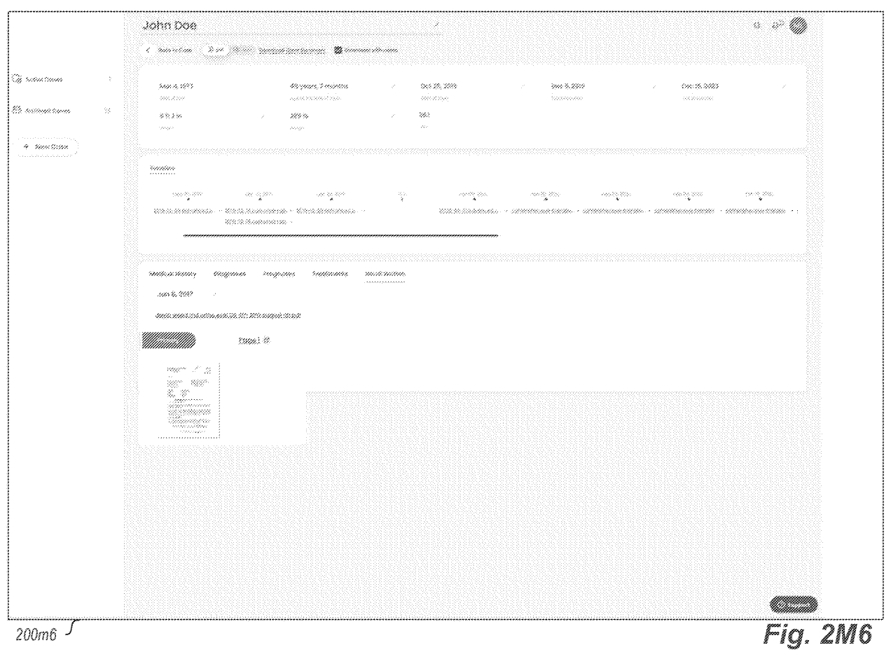

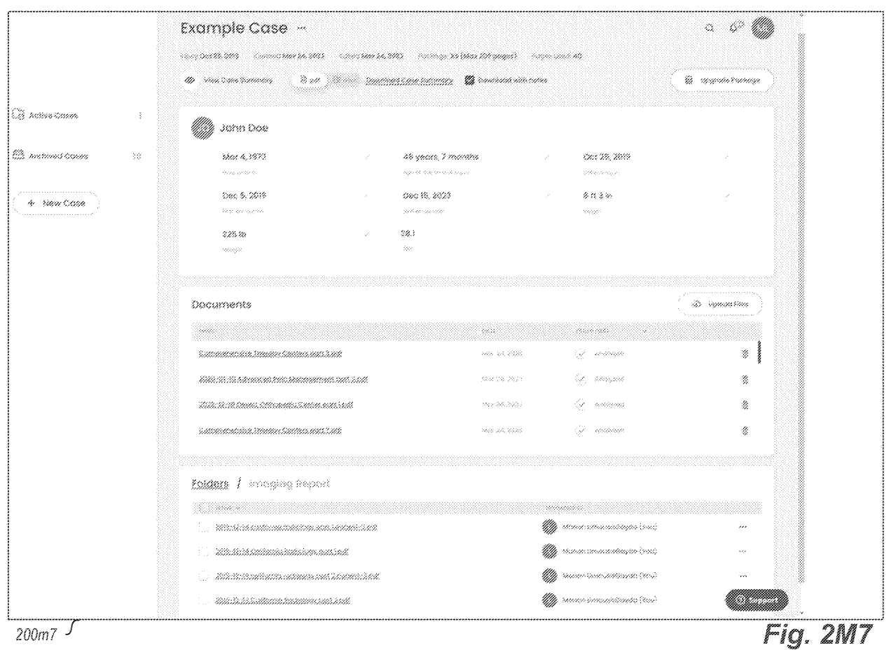

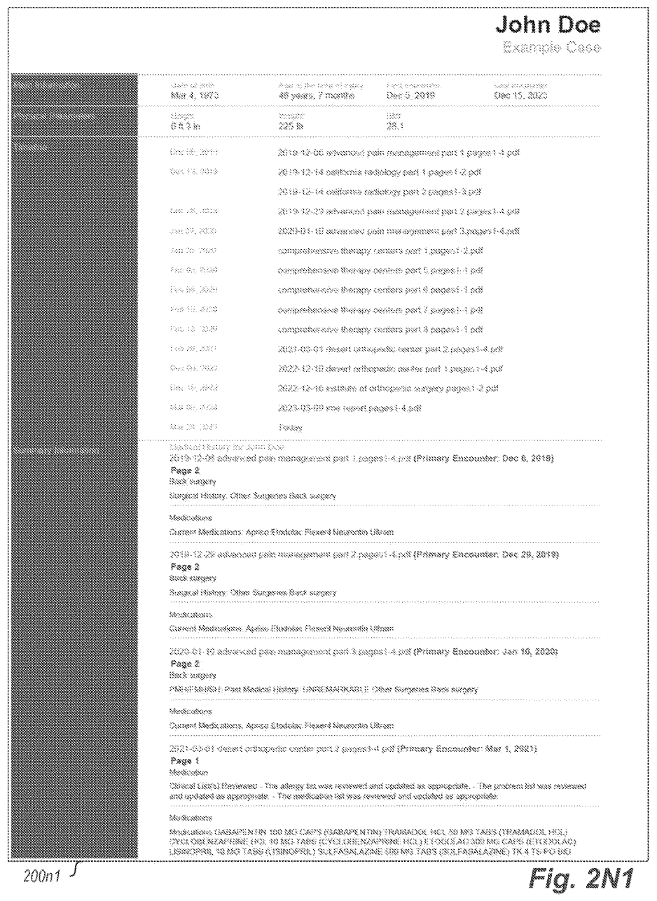

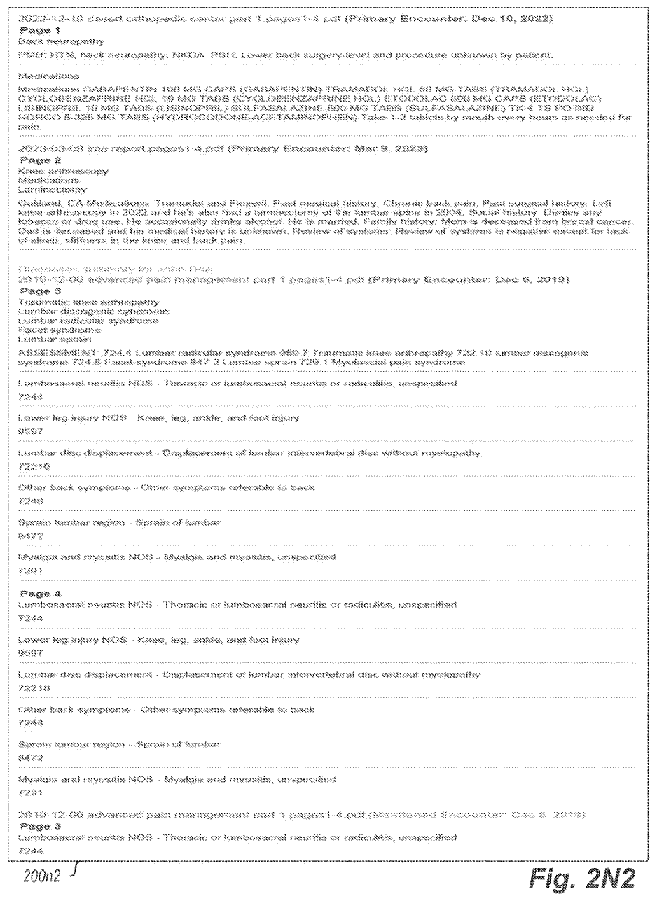

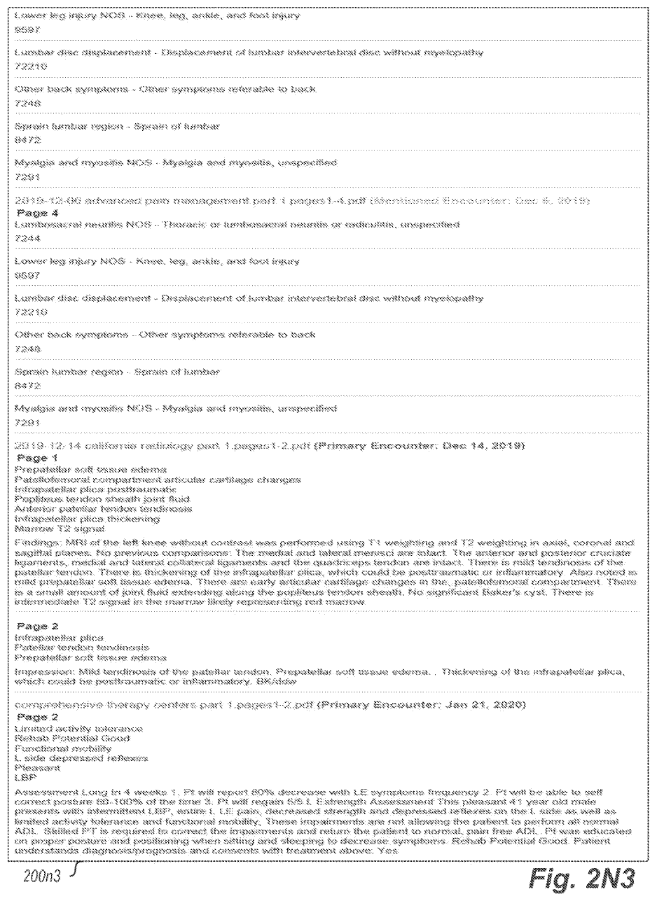

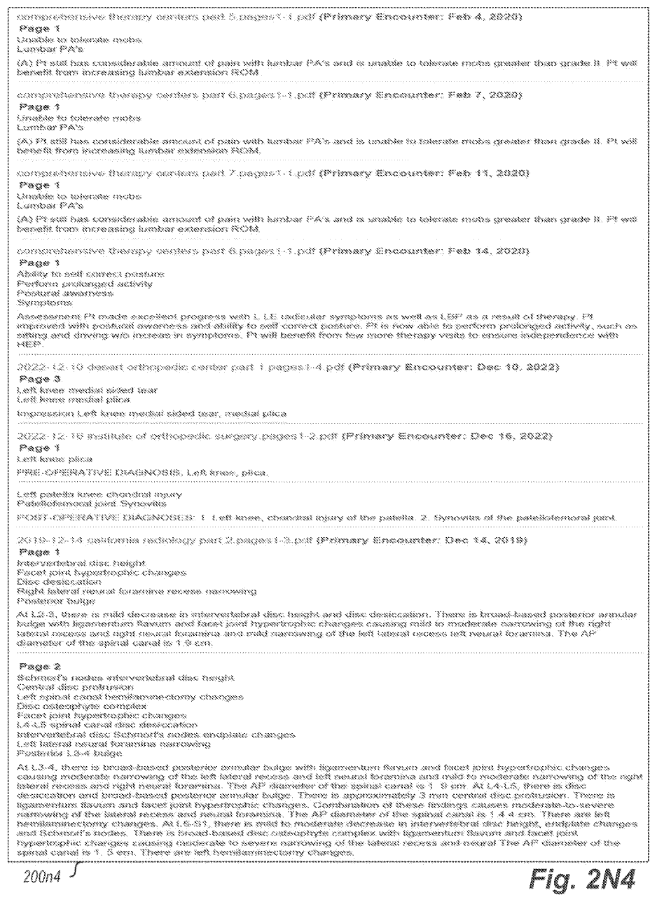

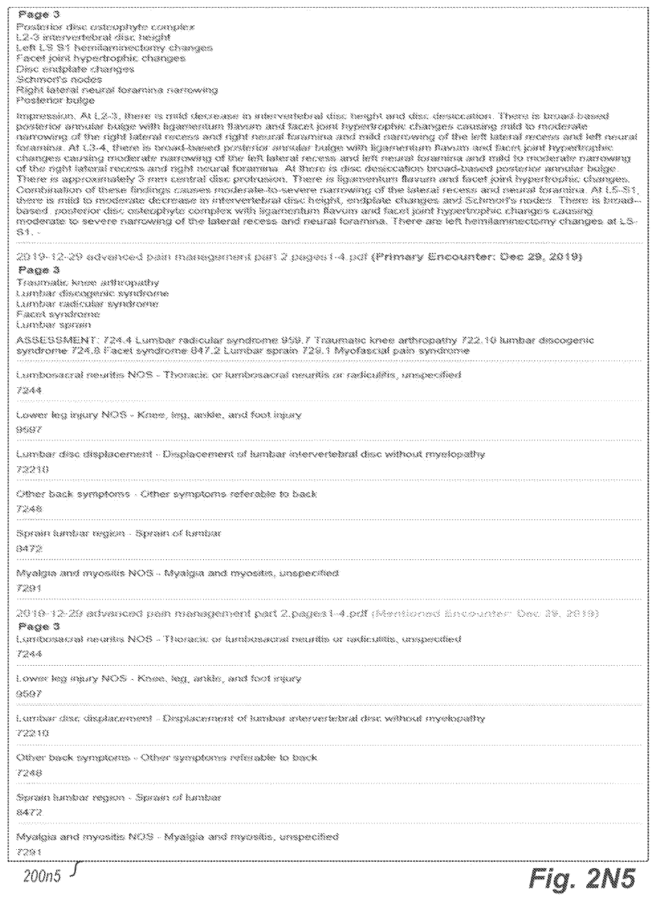

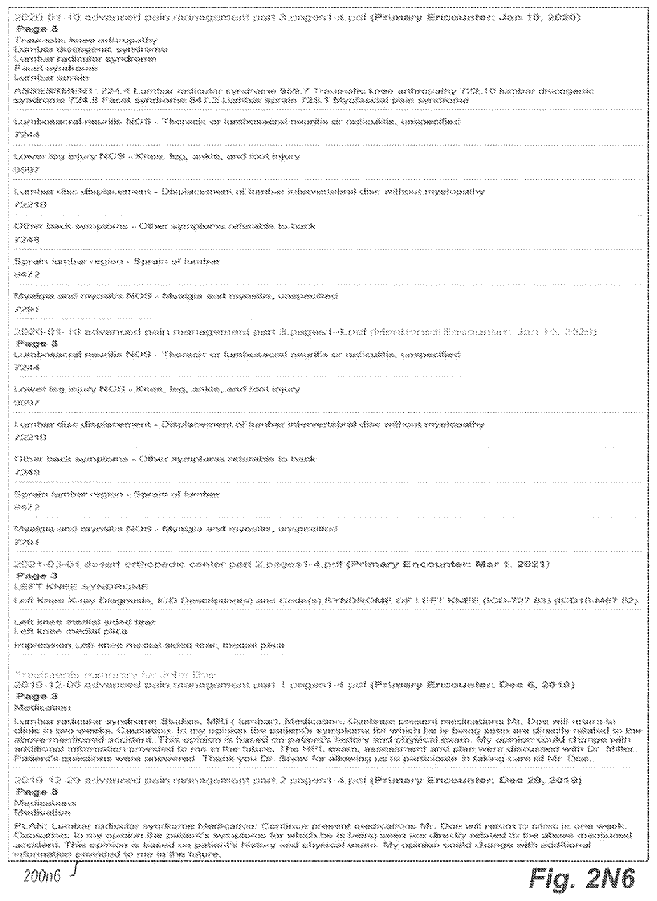

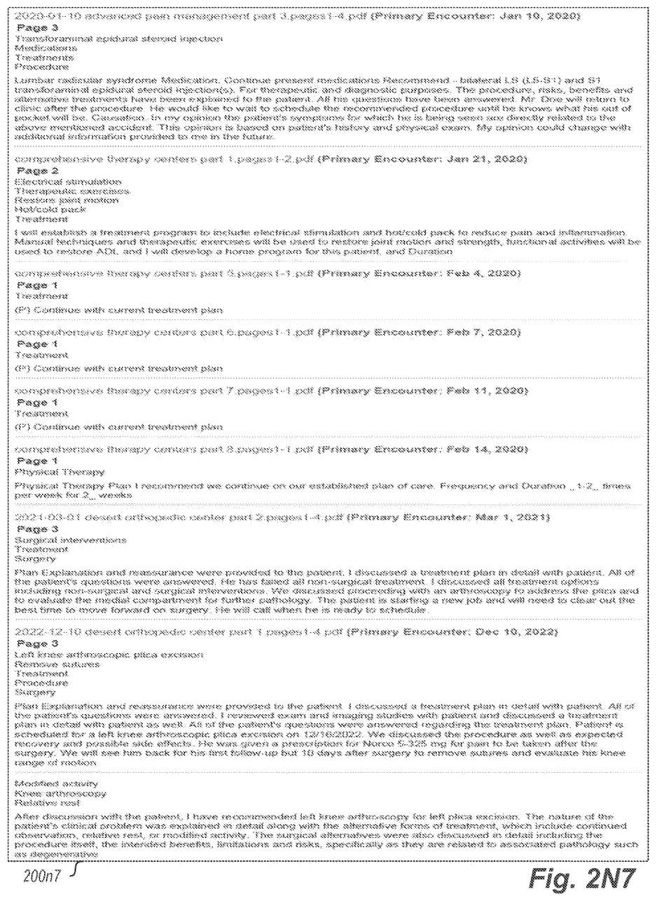

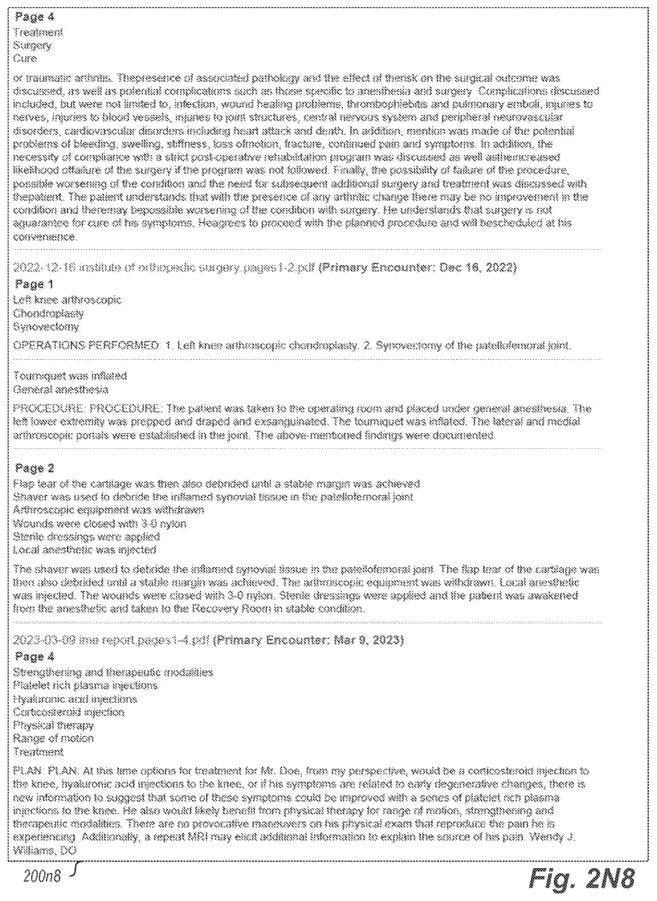

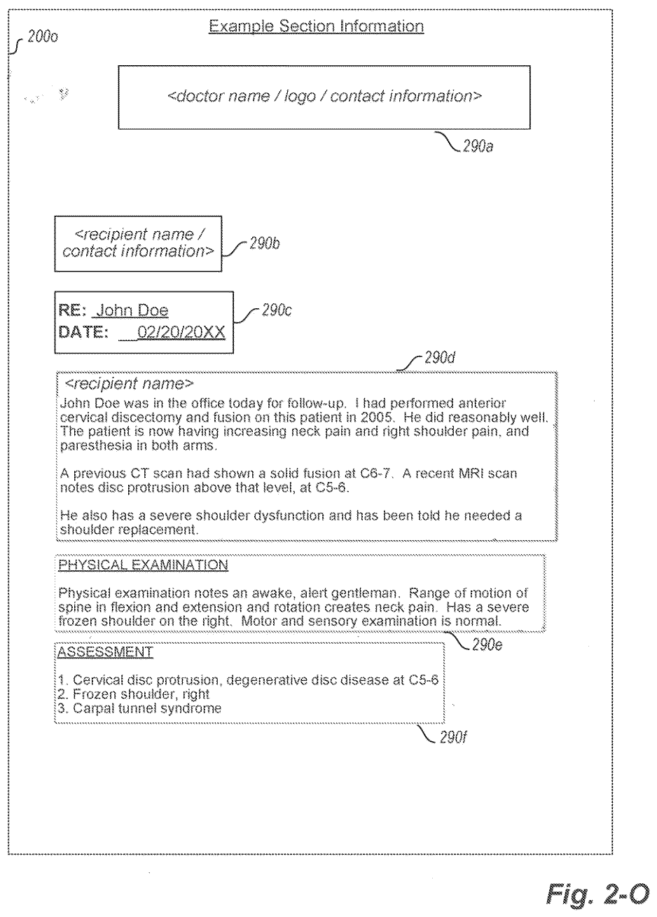

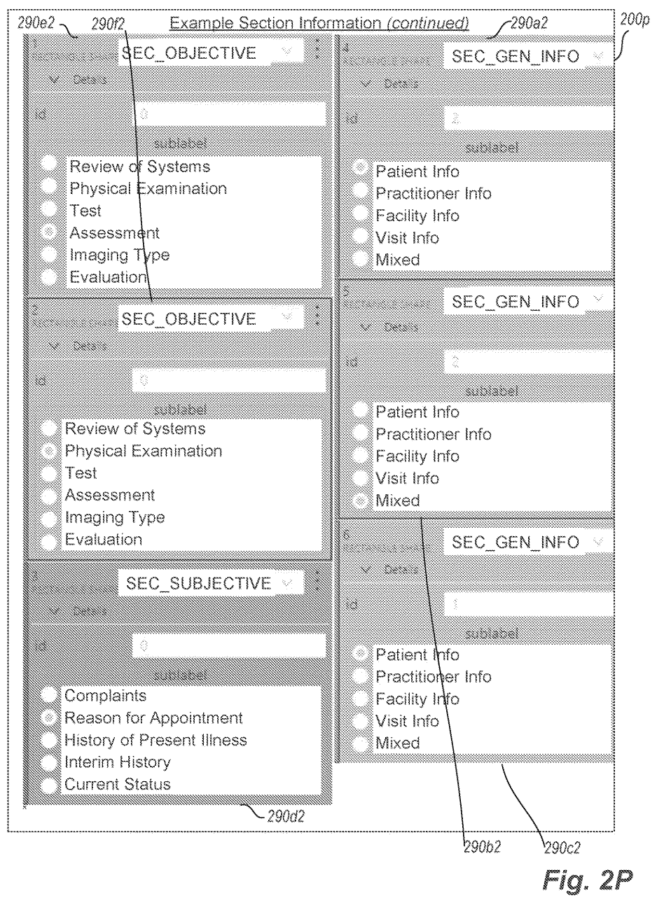

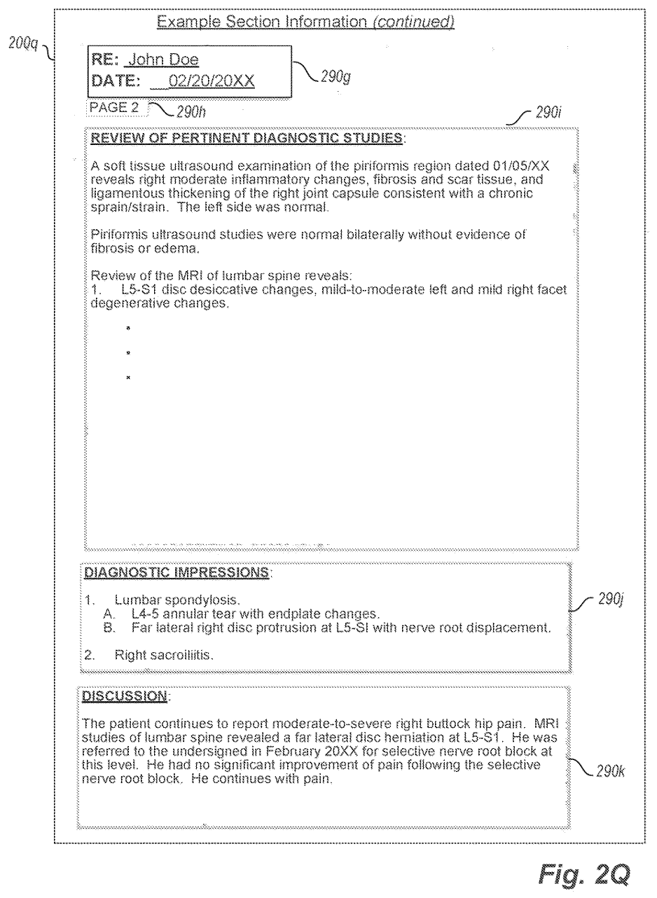

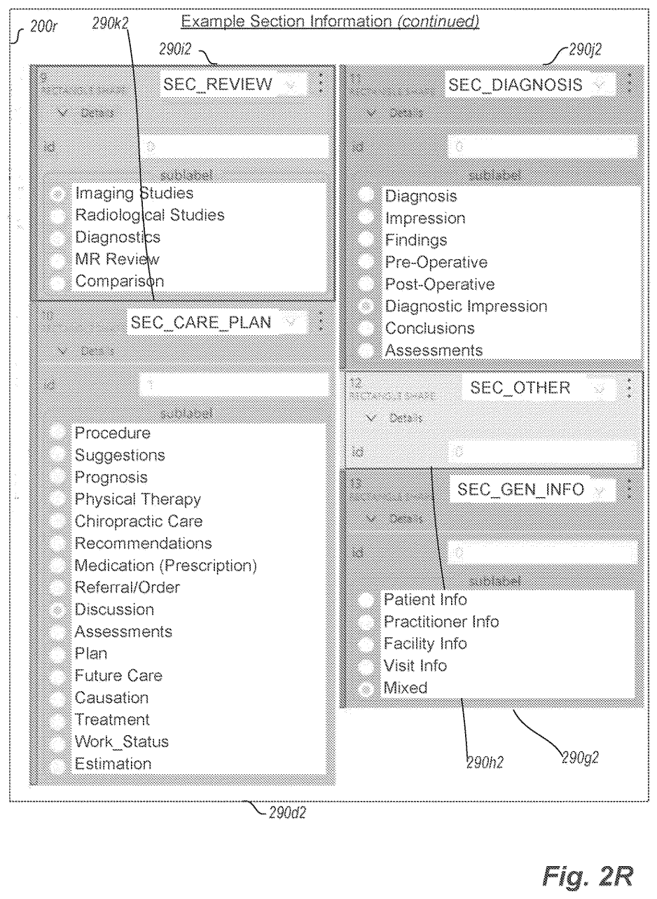

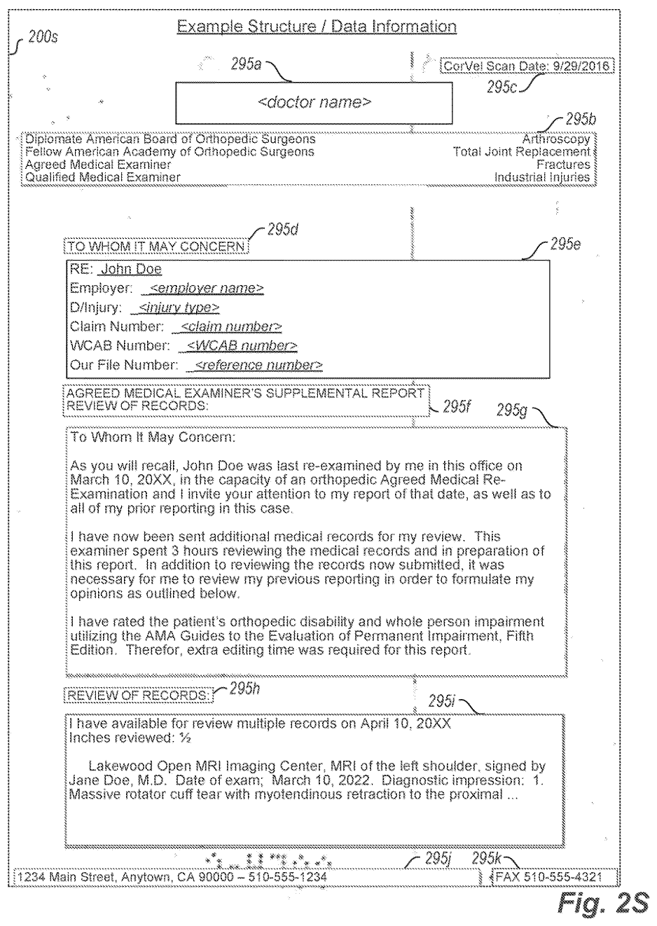

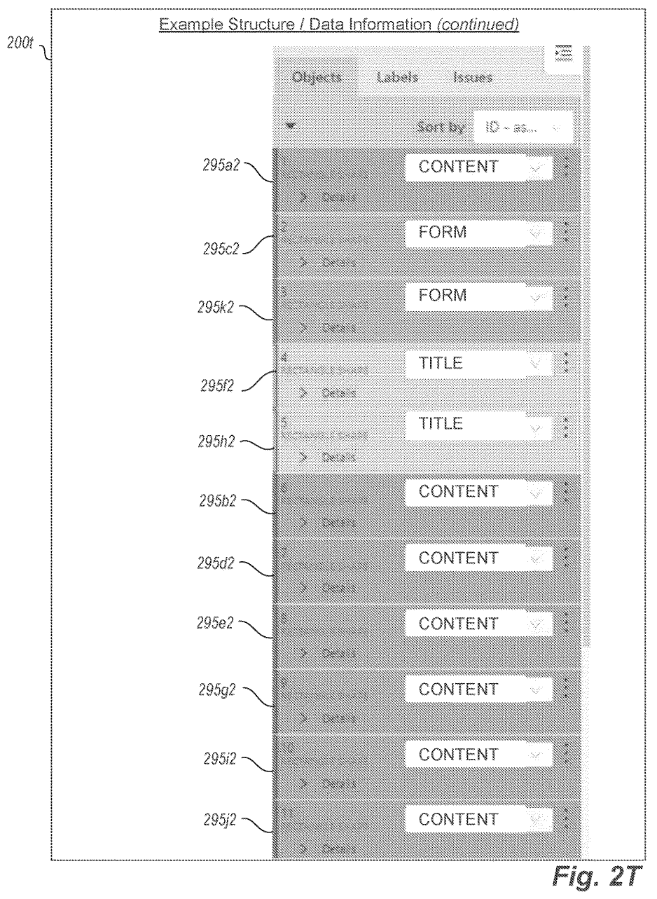

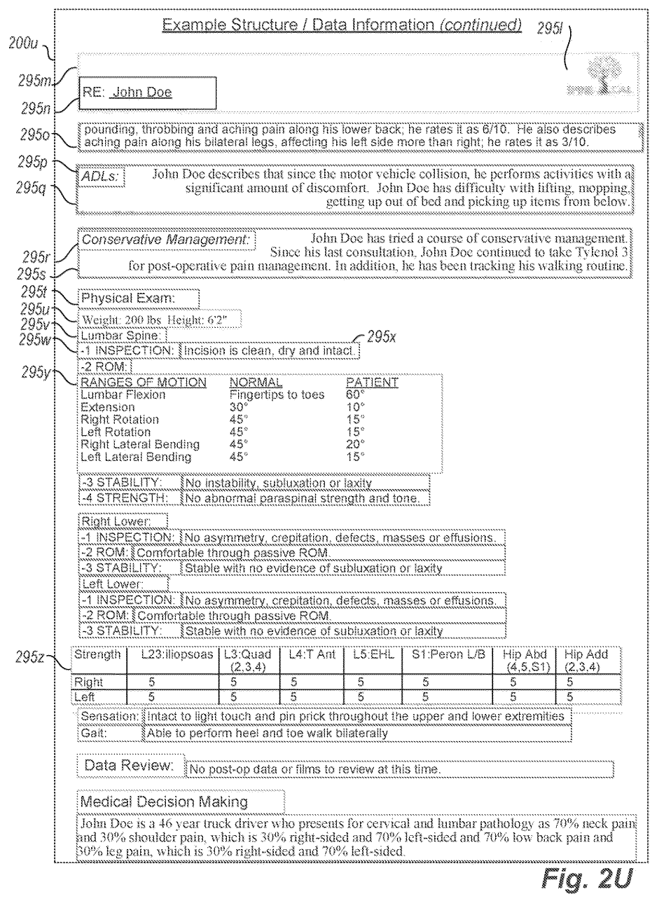

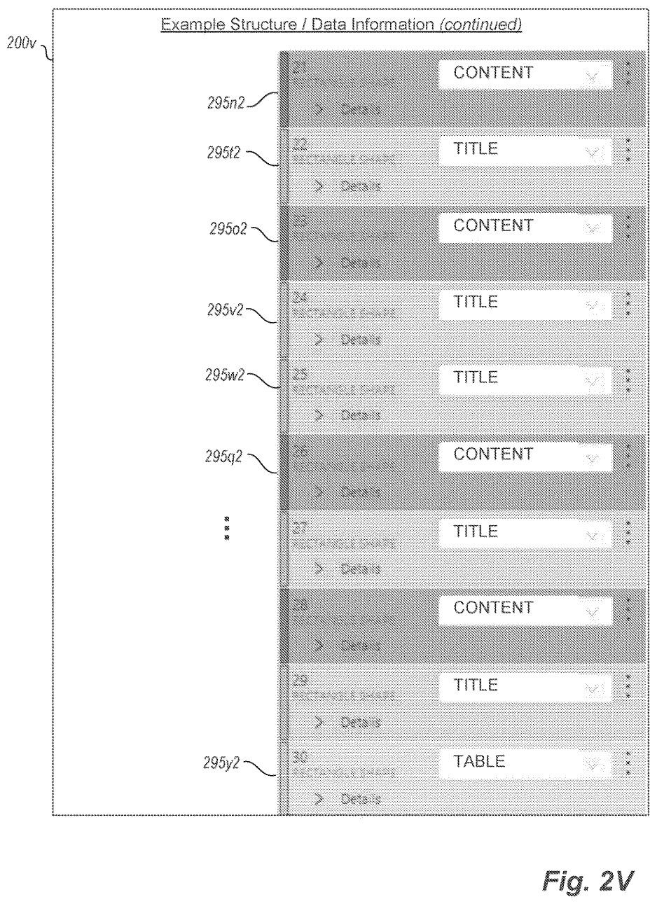

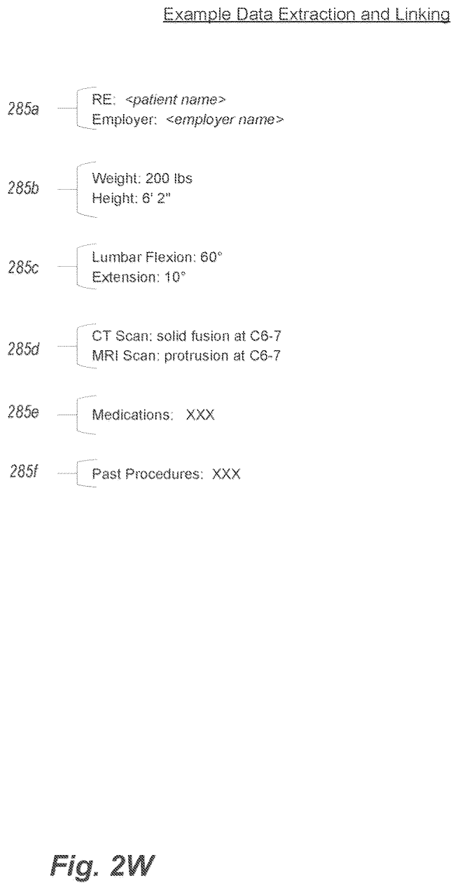

Additional details are included below related to analyzing repair information in various manners, to enable subsequent use of resulting linked repair data, including with respect to non-exclusive examples discussed with respect to FIGS. 2A-2W.

The described techniques may further be used in various manners to address various types of problems. As noted above, in some embodiments the described techniques include identifying repair information related to a particular computing device or particular type of computing device, and in some such cases providing response information that includes information about one or more corrective activities to be performed, optionally in an executable format to initiate one or more automated corrective actions on that particular computing device or on one or more computing devices of that particular type. In other embodiments, the identified repair information may be used in other manners, such as to be provided to one or more users (e.g., the user who supplied a corresponding query) for further use (e.g., to display or otherwise present some or all of the identified repair information to the one or more users), such as for situations in which at least some further user activity is involved (e.g., remove a battery from a smart phone, attach a cable to a specified port, etc.). In some embodiments, the identified repair information may be for types of repair activities or other repair information that does not involve computing devices, such as repair information related to one or more types of medical repair activities (e.g., treatments, procedures, drugs taken, medical devices used, etc.) that have been performed on or taken by an indicated human or other patient entity (e.g., to repair the patient with respect to an indicated medical symptom and/or medical condition of the patient, such as by a type of treatment specified in corresponding indicated repair information; to perform maintenance on the patient, such as with respect to an indicated medical symptom and/or medical condition by performing preventive activities to reduce the likelihood of an indicated medical symptom and/or medical condition arising, etc.) or other medical status for such a patient (e.g., diseases or other conditions of a patient, prior patient activities that affect patient health, etc.)—in such embodiments, the repair information that is summarized and encoded may include, for example, doctors' notes about patients, records of hospitals or other facilities or entities related to treatments (e.g., electronic medical records, or EMRs, from hospitals and/or pharmacies and/or other medical facilities, treatment records from other entities with such information such as insurance companies and/or credit card processing systems, etc.), and additional supplemental information may include information about effects of various activities (e.g., drug interactions, medical guidelines or other information about effects of particular activities, etc.). In addition, in at least some embodiments, the described techniques include automated operations of the ARID system to anonymize or otherwise redact some of the repair information and/or other associated information about particular devices or other entities (e.g., to anonymize patient data to satisfy HIPPA, or Health Insurance Portability and Accountability Act, requirements; to satisfy GDPR, or General Data Protection Regulation, requirements, etc.).

In addition, while various of the discussion herein refers to data groupings that are extracted from “documents”, it will be appreciated that the described techniques may be used with a wide variety of types of content items and that references herein to a “document” apply generally to any such type of content item unless indicated otherwise (explicitly or based on the context), including, for example, textual documents (e.g., Web pages, word processing documents, slide shows and other presentations, emails and other electronic messages, etc.), visual data (e.g., images, video files, etc.), audio data (e.g., audio files), software code, firmware and other logic, genetic codes that each accompany one or more sequences of genetic information, other biological data, etc., and including a grouping of data of multiple such types. Furthermore, the content items may be of one or more file types or other data structures (e.g., streaming data), including document fragments or other pieces or portions of a larger document or other content item, and the contents of such content items may include text and/or a variety of other types of data (e.g., binary encodings of audio information; binary encodings of video information; binary encodings of image information; mathematical equations and mathematical data structures, other types of alphanumeric data structures and/or symbolic data structures; encrypted data, etc.). The group of documents (and/or other content item types) that are used by the ARID system for a particular type of repair information and/or particular target area of interest (referred to generally herein at times as a ‘domain’) may be, for example, all available documents for a particular domain or that includes sufficient documents to be representative of the domain. In some embodiments, each of the documents has contents that are at least partially textual information, while in other embodiments at least some documents or other content items may include other types of content (e.g., images, video information, audio information, etc.).

The described techniques provide various benefits in various embodiments, including to significantly improve the identification and use of responsive information to specified queries, and with such described techniques used in some situations to automatically determine and implement repair activities performed on indicated computing devices or other repair information for such devices. Such automated techniques allow such response information to be generated much more quickly and efficiently than previously existing techniques (e.g., using less storage and/or memory and/or computing cycles) and with greater accuracy, based at least in part on using one or more of the following: the described use of a combination of image-based and textual-based analysis techniques; the described use of domain-specific information to improve and customize the extraction and/or linking of repair for that domain; etc. Non-exclusive examples of additional related benefits of the described techniques include the following: enabling the processing and use of much larger groups of information; enabling identifying responses to queries using information of multiple levels of generality or specificity; enabling identifying responses to queries using data that is combined and linked from multiple separate source documents and/or multiple different repair providers (e.g., for a particular computing device or other entity); etc. In addition, in some embodiments the described techniques may be used to provide an improved GUI in which a user may more accurately and quickly obtain information, including in response to an explicit request, as part of providing personalized information to the user, etc. Various other benefits are also provided by the described techniques, some of which are further described elsewhere herein.

For illustrative purposes, some embodiments are described below in which specific types of information are acquired, used and/or presented in specific ways and by using specific types of automated processing-however, it will be understood that the described techniques may be used in other manners in other embodiments, and that the invention is thus not limited to the exemplary details provided. As one non-exclusive example, while specific types of data structures (e.g., databases, domain-specific data models, language models, etc.) are generated and/or used in specific manners in some embodiments, it will be appreciated that other types of information may be similarly generated and used in other embodiments, including for repair information for areas other than involving computing devices and/or for types of activities other than repair and maintenance actions, and that responses to queries may be used in other embodiments in manners other than automated corrective repair actions, including display or other presentation-for example, in addition to analyzing and using repair data from the types of documents discussed herein in particular examples (including medical records), in some embodiments and situations other types of documents may be analyzed and used (e.g., emails, images, exhibits, etc.) and/or other types of repair data may be analyzed and used (e.g., damage repairs of other types, such as to vehicles and/or other objects; insurance documents; etc.). In addition, various details are provided in the drawings and text for exemplary purposes, but are not intended to limit the scope of the invention. For example, sizes and relative positions of elements in the drawings are not necessarily drawn to scale, with some details omitted and/or provided with greater prominence (e.g., via size and positioning) to enhance legibility and/or clarity. Furthermore, identical or related reference numbers may be used in the drawings to identify the same or similar elements or acts.

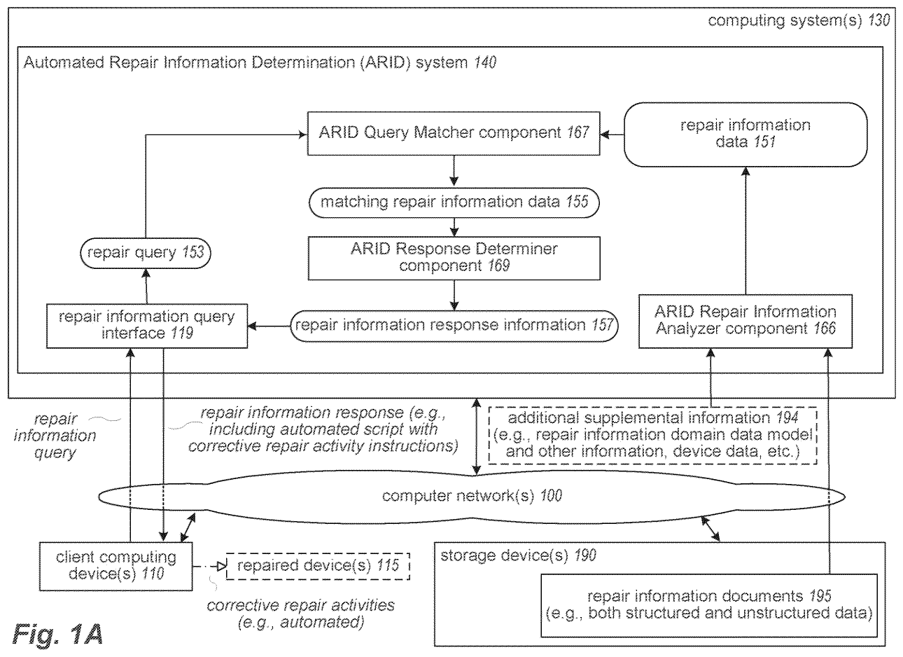

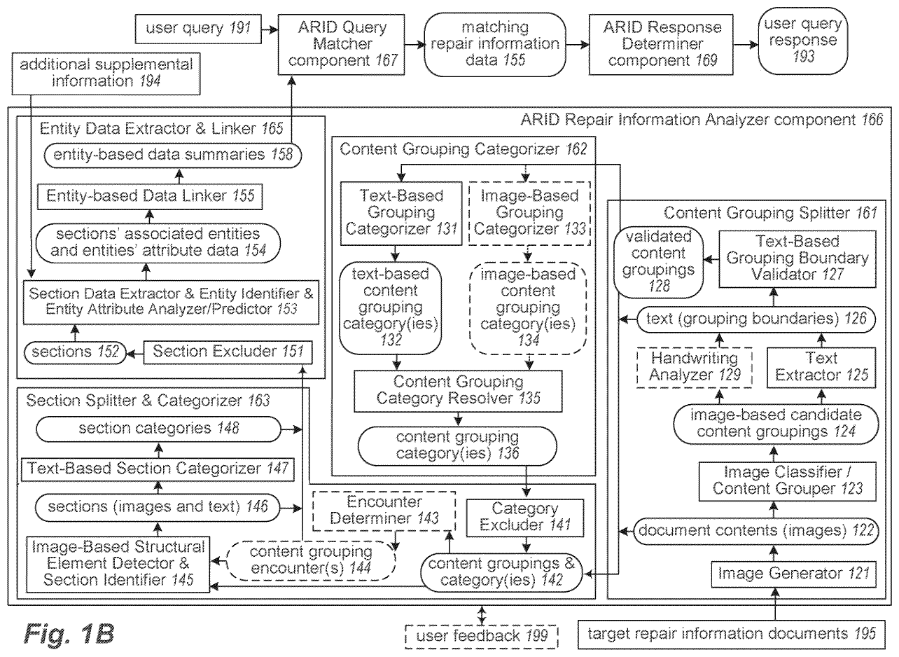

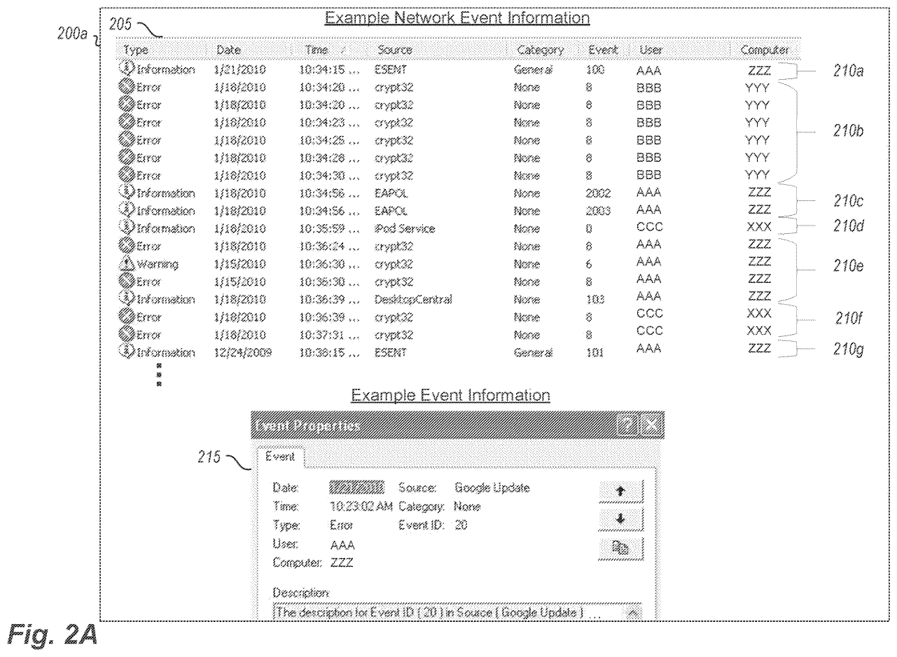

FIG. 1A is a network diagram illustrating an example environment in which a system may be configured and provided for automatically determining repair information for computing devices or other types of repair information, and for providing matching repair-related information in response to queries. In particular, an embodiment of an ARID system 140 is executing on one or more computing systems 130, and includes several components 166, 167 and 169 that generate and use various information 151, 153, 155 and 157.

In particular, as part of the automated operations of the ARID system 140 in this illustrated example embodiment, the system 140 obtains information, such as over the computer network(s) 100, from various repair information documents 195 on one or more storage devices 190 about multiple types of performed repair activities and other repair information for one or more repair domains (e.g., types of computing device repairs). The contents of the repair information documents 195 are received by the ARID system Repair Information Analyzer component 166, which analyzes those contents in order to generate resulting entity-specific repair information 151, which in this example embodiment includes groupings of related data (e.g., stored in SOL tables of one or more SOL databases, not shown) for particular computing device entities and optionally additional expanded content for some or all such data groupings (e.g., additional inferred contents about effects of prior repair activities and/or other associated activities). The repair information 151 is then made available to an ARID system Query Matcher component 167 for further use in addressing queries received from users, and may further be used in other manners (not shown) in other embodiments, whether in addition to or instead of for use in responding to queries. FIG. 1B provides further details about one example embodiment of the ARID system Repair Information Analyzer component 166.

The ARID system 140 operates in an online manner in the illustrated embodiment and provides a graphical user interface (GUI) (not shown) and/or other interfaces 119 to enable one or more remote users (not shown) of client computing devices 110 to interact over the one or more intervening computer networks 100 with the ARID system 140 to obtain functionality of the ARID system. In particular, a particular client computing device 110 may interact over the one or more computer networks 100 with the repair information query interface 119 in order to submit a query about one or more types of repair information (e.g., all available repair information) for an indicated computing device or indicated type of computing device (e.g., corresponding to an associated device 115 for which additional corrective repair activities may be performed, and/or for the client computing device 110 itself), or more generally for repair data matching one or more specified criteria. The resulting repair query 153 is then made available to the ARID system Query Matcher component 167, which compares the repair query 153 to the repair information 151 in order to determine one or more candidate data groupings 155 from the information 151 that match the repair query 153—for example, the ARID system Query Matcher component 167 may use one or more criteria included in or otherwise associated with the repair query 153 to determine matching repair data. The ARID system Response Determiner component 169 then analyzes the matching repair data groupings 155 in order to determine corresponding repair response information 157 for the received query, which it then forwards back to the requesting client computing device 110 in response to the received query via the natural language repair query interface 119. The analysis of the matching repair data groupings 155 may include, for example, analyzing the information of the data groupings to select one or some or all of the data groupings (or portions thereof) to use as the repair response information 157, while in other embodiments all such matching data groupings may be used without such selection, or instead additional information may be added (e.g., adding corresponding information about repair activities to perform to respond to a specific identified repair problem). The repair response information 157 may in some embodiments and situations include executable instructions or other information to automatically cause the recipient client computing device 110 (or an associated target computing device to be repaired, such as a device 115) to execute those repair instructions or to otherwise take automated action to perform repair activities (e.g., corrective repair activities, preemptive additional maintenance activities, etc.). If no data grouping 155 is identified as a response to the received query, the component 169 may instead supply a reply message to the requesting client computing device 110 to indicate that no response is available.

After the requesting client computing device 110 receives the repair response information 157, it may take various actions to use that received information, such as to initiate automated (or other) repair activities on itself or on an associated device 115, and/or may display or otherwise present some or all of the received information to one or more users on the client computing device. The interactions of users and/or client computing devices with the ARID system 140 to obtain functionality of the ARID system may involve a variety of interactions over time, including in some cases independent actions of different groups of users and/or client computing devices.

The network 100 may, for example, be a publicly accessible network of linked networks, possibly operated by various distinct parties, such as the Internet, with the ARID system 140 available to any users or only certain users over the network 100. In other embodiments, the network 100 may be a private network, such as, for example, a corporate or university network that is wholly or partially inaccessible to non-privileged users. Thus, while the ARID system 140 in the illustrated embodiment is implemented in an online manner to support various users over the one or more computer networks 100, in other embodiments a copy of the ARID system 140 may instead be implemented in other manners, such as to support a single user or a group of related users (e.g., a company or other organization, such as if the one or more computer networks 100 are instead an internal computer network of the company or other organization, and with such a copy of the ARID system optionally not being available to other users external to the company or other organizations, such as for a medical facility or a law firm operating a copy of the ARID system to analyze and use confidential medical repair information available for one or more medical patients). In addition, the ARID system 140 and each of its components (including components 166, 167 and 169) may include software instructions that execute on one or more computing systems by one or more processors (not shown), such as to configure those processors and computing systems to operate as specialized machines with respect to performing their programmed functionality. Additional details related to operations of the ARID components 166, 167 and 169 are discussed below with respect to FIG. 1B and elsewhere herein.

FIG. 1B continues the example of FIG. 1A, and illustrates an example embodiment that includes additional example details about the ARID system components 166, 167 and 169 of FIG. 1A, as well as about an embodiment of the ARID system that is capable of providing repair information (or other types of information) for repair areas/domains other than computing devices.

In this example embodiment, the ARID Repair Information Analyzer component 166 receives, from target information documents 195, information about one or more repair areas/domains and/or associated entities, and may further optionally obtain and use additional supplemental information 194 of one or more types as discussed below. The overall goal in this example embodiment is, given documents (e.g., stored in the form of pdfs, images, html, xml, docxs, pptxs, etc.) providing information for one or more devices or other entities and related to a domain, to allow a user to make queries about the domain and obtain responses based on the content contained in the documents (e.g., in a real-time or near-real-time manner with respect to the queries, such as in a matter of seconds or in less than a second). The analysis of the repair documents may use a combination of image-based analysis and text-based analyses, including in some embodiments and situations to use a YOLO (you only look one) architecture to do image-based analyses that detect objects in an image, such as different parts of a document (e.g., structural elements, sections, data values, etc.), and with such information used for further analysis of the documents in the described manners.

In this example embodiment, the information from the target information documents 195 is provided to a Content Grouping Splitter subcomponent 161 of the ARID Repair Information Analyzer component 166, which passes each document to an Image Generator module 121 that generates a corresponding set of images for the document (e.g., one image for each page of the document) if the document is not already in an image format, resulting in images 122 of the document contents. The Image Classifier/Content Grouper module 123 then performs an image-based classification of each document's contents into one or more candidate content groupings 124, such as by predicting whether subsequent images are part of the same or different content groupings. Subsequently, a Text Extractor module 125 extracts text corresponding to some or all of each content grouping that includes typewritten text (e.g., at least portions of each content grouping around boundaries at which content groupings are split), and optionally a Handwriting Analyzer module 129 generates additional text corresponding to handwritten information (if any) of the content grouping, resulting in text 126 corresponding to at least the content grouping boundaries (and optionally all text in each content grouping)-the generated textual data may be extracted from the original source documents 195 if they include that textual data in a readable format (e.g., pdfs, html, xml, docxs, pptxs, etc.) and/or from images (e.g., the document contents 122) via optical character recognition (OCR) or other text identification techniques. A Text-Based Grouping Boundary Validator module 127 then analyzes the text 126 to validate the image-based candidate content groupings 124 (or to modify candidate content groupings determined to be incorrect), resulting in validated content groupings 128 (optionally each including the images 122 and/or text 126 corresponding to that content grouping). Additional details related to nonexclusive examples of performing content grouping splitting are discussed further with respect to FIGS. 2A-2W.

Output of the Content Grouping Splitter subcomponent 161 is then provided to a Content Grouping Categorizer subcomponent 162, including information about the validated content groupings 128 (e.g., including extracted text and/or images for each content grouping). In the illustrated embodiment, the Text-Based Grouping Categorizer module 131 then performs a text-based analysis of the content of each content grouping to determine one or more corresponding categories 132 for the content grouping, which in some embodiments are then stored as one or more final content grouping categories 136 for each content grouping. In some embodiments, an optional Image-Based Grouping Categorizer module 133 also performs an image-based analysis of the content of each content grouping to determine one or more corresponding categories 134 for the content grouping, such as in parallel with the module 131—if both categories 132 and 134 are generated, a Content Grouping Category Resolver module 135 then determines the one or more final content grouping categories 136 for each content grouping by combining the information about the categories 132 and 134 (e.g., in a weighted manner). Additional details related to nonexclusive examples of performing content grouping categorizing are discussed further with respect to FIGS. 2A-2W.

Output of the Content Grouping Categorizer subcomponent 162 is then provided to a Section Splitter & Categorizer subcomponent 163, including the content grouping categories 136, along with the validated content groupings 128 (and corresponding extracted images 122 and text 126) to which the categories correspond. In the illustrated embodiment, information about the content grouping categories 136 are provided to a Category Excluder module 141 of the subcomponent 163, which excludes certain content grouping categories from further analysis, resulting in remaining content groupings and categories 142 to be provided for further analysis by the subcomponent 163—in other embodiments, no such category-based exclusion may be performed, or instead exclusion of some content groupings may be performed in other manners. In the illustrated embodiment, an Encounter Determiner module 143 may optionally be provided and used to further determine one or more encounters in each content grouping from information 142, resulting in content grouping encounters 144—if so, such information 144 is further provided to module 145 for further analysis along with the information 142, and if not then just the information 142 is provided to module 145 for further analysis. The Image-Based Structural Element Detector & Section Identifier module 145 then performs an image-based analysis of each content grouping from information 142 or each encounter from information 144 in order to detect structural elements within that information, determine a type or topic of each such structural element, and combine at least some such structural elements together to form related sections (while optionally treating other identified structural elements as separate sections), resulting in sections 146 (e.g., including the images and/or text for each such section that are part of the contents of that section). A Text-Based Section Categorizer module 147 then performs a text-based analysis of each section in order to determine one or more categories for each section, resulting in section category information 148. Additional details related to nonexclusive examples of performing section splitting and categorizing are discussed further with respect to FIGS. 2A-2W.

Output of the Section Splitter & Categorizer subcomponent 163 is then provided to an Entity Data Extractor & Linker subcomponent 165, including the sections 146 and categories 148. In the illustrated embodiment, the information 146 and 148 is provided to a Section Excluder module 151 of the subcomponent 165, which excludes certain section categories from further analysis, resulting in remaining sections and categories 152 being provided to a Section Data Extractor & Entity Identifier & Entity Attribute Analyzer/Predictor module 153—in other embodiments, no such section-based exclusion may be performed (such that the sections and categories 152 include all sections 146 and their respective categories 148), or instead exclusion of some sections may be performed in other manners. The module 153 then performs automated operations to extract data from each remaining section, including to identify an associated entity, and to predict or otherwise determine values for attributes of the entity, resulting in each section's associated entities and entity attribute data 154. As part of doing so, the module 153 may obtain and use various types of additional supplemental information 194, such as a language model specific to the type of repair data being analyzed (e.g., a trained Bio-Bert model for medically related data), information about specific entities having repair information in the documents 195 (e.g., device recalls; demographic information for particular people, such as medical patients; etc.), and optionally other types of related information (e.g., information about drug interactions; time-based effects of particular activities, such as smoking; expected results from particular medical procedures or other treatments, such as from medical clinical guidelines; etc.)—in addition, the module 153 may further perform processing to identify various parts of the content, such as particular repair activities, particular conditions to be repaired or ameliorated, particular objects used for repair activities (e.g., medical devices, drugs, etc.), particular repair-related tests that are performed, etc., optionally based on or associated with particular codes for a particular domain (e.g., for the medical domain, CPT, or Current Procedural Terminology codes; ICD, or International Classification of Diseases, codes; HCPCS, or Healthcare Common Procedures Coding System, codes; etc.), and particular attributes associated with some or all of the other identified parts (e.g., associated timing information, such as start and/or end dates, whether using absolute times or times relative to another indicated time, such as a date or other time associated with a document or a particular entry in the document; a level of a repair activity performed, such as a dosage and/or strength and/or frequency of medicine used or other medical procedure or intervention; results of repair-related tests; etc.). The information 154 is then supplied to an Entity-Based Data Linker module 155, which links together extracted data for one or more particular entities from multiple documents 195 and/or content groupings 128 and/or sections 152, resulting in entity-based data summaries 158, such as by using identifying information to link data from different documents or other groups of information about the same entity (e.g., a unique device identifier for a device, such as a MAC address or persistent IP address; a social security number or patient number or patient name or other identifying number for a person who is a medical patient; etc.). The information 158, and other information generated by the component 166, may be stored persistently in one or more manners (e.g., in one or more databases on one or more storage devices, not shown), and in this example embodiment is further made available to the ARID Query Matcher component 146. In addition, feedback 199 of various types may optionally be provided to and used by the component 166, such as modify automatically generated data (e.g., to correct errors, to add supplemental information, etc.). Additional details related to nonexclusive examples of performing data extraction and linking are discussed further with respect to FIGS. 2A-2W.

The ARID system may further receive one or more user queries 191 that are supplied to the ARID Query Matcher component 167, which identifies candidate matching repair information data 155 from the generated information 158 (e.g., a best matching set of one or more linked data groupings; all matching sets of linked data groupings, such as for a particular indicated entity; a top N number of candidate sets of linked data groupings, with N being customizable or a fixed number; etc.), and provides the matching data 155 to the ARID Response Determiner component 169, which generates and provides a corresponding user query response 193. The user query response 193 may, for example, correspond to information 157 of FIG. 1A In some embodiments, a query response 193 may, for example, include indications of additional non-textual information (e.g., images, audio, etc.) in one or more documents from which the data grouping is extracted (e.g., by including links in the provided response to the corresponding parts of that document, by extracting and bodily including that additional non-textual information, etc.).

While a variety of details have been discussed with respect to the example embodiments of FIGS. 1A-1B, it will be appreciated that other embodiments may not include some such details, and/or may include other functionality that is not illustrated in these examples.

FIGS. 2A-2W illustrate further examples of performing described techniques, including automatically determining repair information for use in responding to queries and/or in other manners.

With respect to FIG. 2A, it illustrates example information 200a corresponding to network event information that may indicate repair problems and/or associated repair activities for one or more computing devices. In particular, information 205 illustrates example network event information (e.g., from an event log), which in this example may be separated into various encounters 21Oa-21Og corresponding to different associated computing device entities XXX, YYY and ZZZ with associated user entities CCC, BBB and AAA, respectively. It will be appreciated that some encounters 210 correspond to a single event, such as for encounter 21Oa, while other encounters may correspond to a series of related events, such as for encounter 21Ob. Information 215 further provides example additional data available for each event, such as to include information about an associated time (in this case, with both a date and a time of day), a type, a unique ID, a source, descriptive text, etc. it will be appreciated that the types of event information may vary in other embodiments and situations, and may be formatted in different manners than is illustrated.

FIG. 2B continues the example of FIG. 2A, and illustrates additional example information 200b about repair activities performed for a particular associated computing device entity, and in particular illustrates example maintenance activities performed for that computing device. In this example, the maintenance activities may be separated into multiple encounters 220a-220d corresponding to different associated repair/maintenance sessions, with some such encounters 220 corresponding to a single repair activity, such as for encounter 220a, while other encounters may correspond to multiple related repair activities, such as for encounter 220d. In this example, a provider of the repair activities is not identified, and may correspond to a single provider, although in other embodiments and situations multiple providers may perform different repair activities (e.g., at different times) for the associated computing device.

FIG. 2C continues the examples of FIGS. 2A-2B, and illustrates additional example information 200c about repair activities performed for a particular associated computing device entity by a particular provider of repair activities, and in particular is part of an invoice generated from that provider. In this example, information 200c includes various structural elements and associated sections, such as with the “Details” section including overview information, a “From” section including information about the provider, a “Bill To” section including information about the recipient (e.g., a user or company associated with the computing device), a table section “Service” that provides information about repair activity services performed in a tabular format, a table section “Parts/Materials” that provides information about repair objects (in this case parts and materials) used as part of the repair activity services, a “Notes” section that provides handwritten information with further details about the repair activities performed, etc.

While various example information has been provided in FIGS. 2A-2C related to types of repair information that may be available and used for computing device entities, it will be appreciated that other types of repair information may be available and used in other embodiments.

FIGS. 2D-2W provide further examples of automatically determining repair information for use in responding to queries and/or in other manners, including for repair information corresponding to medical repair activities for associated patient entities (e.g., by corresponding medical professional providers of medical services). For example, FIGS. 2D-2F illustrate examples of particular repair information documents that may be provided from a first provider of medical services for a particular patient entity, while FIGS. 2G-2L2 illustrate examples of additional repair information documents for that patient entity from other providers of medical services, FIG. 2M1-2N8 illustrate examples of a GUI (graphical user interface) for accessing an entity's repair-related information, and FIG. 2-O through -2W illustrate examples of associated analysis activities performed by the ARID system for example repair information documents.

In particular, FIG. 2D includes information 200d corresponding to a transmittal cover page (in this example, a fax coversheet) from a medical provider to a recipient (e.g., a user authorized to receive and review a particular patient entity's medical records, such as a lawyer involved in a related lawsuit), indicating that the following pages of the transmittal include medical records and an associated bill. When analyzed by the ARID system, the information 200d may be identified as a separate content grouping from the next page of the transmittal (shown in FIG. 2E) that includes medical record information (e.g., as part of a second content grouping that corresponds to the information shown in FIG. 2E and additional related information, not shown), and the information 200d may be categorized as being of a type that is excluded from further analysis (e.g., due to such transmittal documents not typically including substantive medical repair data). FIG. 2E continues the example of FIG. 2D, and illustrates a first of multiple pages of medical records that in this example correspond to a particular encounter with a patient by the medical services provider, and that may further include a variety of types of medical data about the patient (e.g., demographic information, past treatment history, current symptoms, current treatments, etc.). In particular, FIG. 2E illustrates information 200e that includes information about the patient name and date, and the beginning of a physical examination form with information about a date of an incident that resulted in injuries, including corresponding symptoms-while additional pages of the physical examination form are not illustrated, examples of such additional pages are included in U.S. Provisional Patent Application No. 63/331,414, filed Apr. 15, 2022 and entitled “Determining Repair Information Via Automated Analysis Of Structured And Unstructured Repair Data”. FIG. 2F continues the examples of FIGS. 2D-2E, and illustrates continuing information 200f from the encounter, including billing information related to medical activities performed. In a manner similar to that of the transmittal information shown in FIG. 2D, the billing information illustrated in FIG. 2F may be identified as a separate content grouping relative to the medical notes illustrated in FIG. 2E and may be categorized in a manner that excludes it from further analysis due to the billing information, or alternatively the list of medical activities performed may be extracted and used as part of the medical data generated for the patient entity from the encounter. The information of FIGS. 2E-2F and of other additional pages of the physical examination form (not shown, such as to include information about medical treatments performed, further details of the symptoms of the patient, a past medical history for the patient with details such as medications and supplements taken, past vitals measurements, results of medical tests performed on the patient, etc.) may be determined to be part of the same content grouping in various manners, such as based on the similarity of the information formatting (e.g., similar headers and/or footers, similar type faces and layout, etc.), sequential page numbers, the same date and patient name, etc. FIGS. 2G-2J continue the examples of FIGS. 2D-2F and illustrate additional information about a further patient encounter with another medical provider, including information 200g of FIG. 2G that shows information about the patient name and date, and the beginning of a physical examination form with information about a date of an incident that resulted in injuries, including corresponding symptoms, and with information 200h, 200i and 200j of FIGS. 2H, 21 and 2J, respectively, including additional related information, such as a past medical history, measurements, exam results, assessment results, a treatment plan, and codes summarizing the diagnoses and corresponding activities-as with the information of FIGS. 2E-2F, the information of FIGS. 2G-2J may be determined to be part of the same content grouping and as part of a single encounter in various manners. FIG. 2K continues the examples of FIGS. 2D-2J and illustrates additional information 200k about a further patient encounter with another medical provider, including information about the patient name and date, and findings from an MRI procedure on the left knee. FIG. 2L1 and 2L2 continue the examples of FIGS. 2D-2K, and illustrate information 200l1 and 200l2, respectively, that correspond to an encounter by the same patient with yet another medical provider, which in this example relates to an acupuncture treatment. It will be appreciated that the data of FIGS. 2D-2L2 are provided for purposes of example, and that the invention is not limited to the details shown.

With respect to determining content groupings and/or encounters, the described techniques may include analyzing contents of a document having repair data (and optionally additional types of data) using one or both of image-based analysis and text-based analysis to identify one or more content groupings each having related content (e.g., separate sub-documents that were combined to form the document, content of different types, content that is generated at different times and/or in different manners, etc.) and/or one or more encounters each corresponding to a single repair or other interaction session. For example, a trained neural network model may be used to perform a pairwise analysis of pairs of sequential images (e.g., images of separate pages) in order to determine whether the content of those two images are part of the same or different content groupings (i.e., whether or not a split exists between two different content groupings to which those two images correspond, such as a split occurring between two corresponding pages) and/or are part of the same or different encounters (i.e., whether or not a split exists between two different encounters to which those two images correspond, such as a split occurring between the two corresponding pages), and a trained transformer neural network model may be used to analyze text surrounding each determined split point to validate (or overrule) the initial split determination. Once the separate content groupings and/or encounters are identified, each such separate content grouping and/or encounter may then be analyzed to categorize that content grouping (e.g., from an enumerated list or set of content grouping categories) and/or encounter (e.g., from an enumerated list or set of encounter categories), including in at least some embodiments to use a combination of image-based analysis and text-based analysis, such as by a trained transformer neural network model that initially classifies the content of that content grouping or encounter as belonging to a particular content grouping category or encounter category based on the text of the content grouping or encounter (e.g., using only a subset of the content, such as 1-3 pages), and optionally with a subsequent image-based analysis of the content to validate (or overrule) that initial categorization (e.g., using a trained neural network model).

FIG. 2O (referred to herein as ‘2-O’ for the sake of clarity) through 2R continue the examples of FIGS. 2D-2L2, and illustrate section information determined for an example set of medical data 2000 and 200q corresponding to a single encounter and content grouping, although in other embodiments and situations a single document and/or content grouping and/or page may include information about multiple encounters. In this example of FIG. 2-O, various sections 290 are identified in information 2000, with information 200p of FIG. 2P providing further details about the sections-for example, a header section 290a is identified and may be excluded from further analysis, while other content sections 290b-290f are identified and may be further analyzed to extract relevant medical data. As further illustrated in FIG. 2P, additional data may be determined for each section, such as with a categorization label to correspond to a type of category of the section as illustrated in information sets 290a2-290f2 corresponding to sections 290a-290f, respectively. FIG. 20 continues the examples of FIGS. 2D-2P, and in particular illustrates a second page of information 200q following that beginning in information 2000 of FIG. 2-O, with the information 200q being part of the same encounter and content grouping as that of information 2000, and including additional identified sections 290g-290k. In this example, section 290h may be classified as a form that does not have substantive medical data for further analysis (but may be used as part of determining that the ‘page 2’ of information 200q is part of the same content grouping and encounter as information 2000 of the preceding first page), while the other sections of information 200q may be content sections with relevant medical data to be extracted and further used. The information sets 290g2-290k2 of FIG. 2R correspond to the sections 290g-290k of FIG. 20, respectively, and illustrate additional information in a manner similar to that discussed with respect to FIG. 2P. Each section may be determined based at least in part on identified structural elements, as discussed further with respect to FIGS. 2S-2V. As non-exclusive examples, section types and associated values may include some or all of the following: review information, with values including “Imaging Studies”, “Radiological Studies”, “Diagnostics”, “MR Review”, “Comparison”, etc. ; past medical history information, with values including “Past Medical”, “Allergies”, “Medications”, “Social”, “Surgical”, “Family”, “Tobacco/Alcohol/Supplement”, “Serious Conditions”, “Previous Injuries”, “Radiological Testing”, “Summary”, etc. ; diagnosis information, with values including “Diagnosis”, “Impression”, “Findings”, “Pre-Operative”, “Post-Operative”, “Diagnostic Impressions”, “Conclusions”, “Assessment”, etc. ; care plan information, with values including “Procedure”, “Suggestions”, “Prognosis”, “Physical Therapy”, “Chiropractic Care”, “Recommendations” “Medication (Prescription)” “Referral/Order” “Discussion”, “Assessments”, “Plan”, “Future Care”, “Causation”, “Treatment”, “Work_Status”, “Estimation”, etc. ; general information, with values including “Patient Info”, “Practitioner Info”, “Facility Info”, “Visit Info”, “Mixed”, etc. ; objective information, with values including “Review of Systems”, “Physical Examination”, “Test”, “Assessment”, “Imaging Type”, “Evaluation”, etc. ; subjective information, with values including “Complaints”, “Reason for Appointment”, “History of Present Illness”, “Interim History”, “Current Status”, etc. ; prognosis information; other information; etc.

FIGS. 2S-2V continue the examples of FIGS. 2D through 2L2 and 2-O through 2R, and illustrate structural element information determined for an example set of medical data 200s and 200u corresponding to an encounter and content grouping. In the example of FIG. 2S, various structural elements 295a-295k are shown in information 200s, and additional corresponding structural elements 2951-2952 are shown in information 200u of FIG. 2U. The information sets 295a2-295k2 of FIG. 2T correspond to the structural elements 295a-295k, respectively, of FIG. 2S, and the information sets 29512-29522 of FIG. 2V correspond to the structural elements 2951-2952, respectively, of FIG. 2U. As illustrated in FIGS. 2T and 2V, information about the various structural elements may be determined and stored in a manner similar to that previously discussed with respect to identified sections, and various types of medical data may be extracted from at least some of the structural elements. The structural elements may be of various types, such as tables 295y and 295z of FIG. 2U (whether with or without lines to indicate rows and columns), forms with key value pairs 295u, textual medical data such as in structural element 295x, etc. The structural elements may be further joined together into corresponding sections, such as the section including structural subtitle and content elements 295r and 295s, as discussed elsewhere herein. As non-exclusive examples, structural elements may include some or all of the following: “subtitle”; “content”; “form”; “table”; “header”; “footer”; “title”; “questionnaire”; “other”; etc.

With respect to identifying structural elements and sections, in at least some embodiments, some or all content groupings (e.g., content groupings remaining after the optional exclusion of some content grouping categories) or encounter groupings (if available) are each analyzed to identify subsets of that content that include structural elements, by using a trained computer vision object-detection model (e.g., Detectron2) to analyze images of that content and determine separate structural element objects of that content, such as to identify titles, sub-titles, tables, ‘forms’ with key-value pairs, questionnaires, header/footers, images, graphs, substantive data (e.g., text, numbers, etc.), paragraph and line numbers, etc., and to optionally assign corresponding labels to each structural element related to the type of the structural element. Sections may then be identified by, for example, combining related structural elements (e.g., based at least in part on structural element labels/types, such as to combine a title and/or sub-title with following substantive data), while treating other structural elements (e.g., tables, forms, questionnaires, images, graphs, etc.) as separate sections. Once the separate sections are identified, each such separate section is then analyzed to categorize that section (e.g., from an enumerated list or set of section categories), including in at least some embodiments to use a text classification model (e.g., a trained transformer neural network model, such as based on a Bert NLP, or Natural Language Processing, model; a text vectorization technique; etc.) that classifies the content of that section as belonging to a particular section category based at least in part on text of that section.

FIG. 2W continues the examples of FIGS. 2D-2V, and illustrates example data 285a-285d that may be extracted and linked from various other medical documents, including in this example to have general information 285a, key-value pairs 285b, test results 285c, medical procedures performed 285d (and resulting information), medications 285e, medical history information 285f, etc. With respect to extracting repair data from a section or structural element into one or more data groupings corresponding to an associated entity, in at least some embodiments, some or all sections (e.g., sections remaining after the optional exclusion of some section categories) and/or their structural elements are each analyzed to extract repair data from the content and to identify an associated device or other entity to which the repair data corresponds (e.g., the device or other entity on which the repair activities are performed), such as to identify particular repair activities performed and corresponding results for a particular associated device or other entity, including to determine values for particular attributes of the associated device or other entity. For example, text may be extracted and analyzed in at least some embodiments using one or more trained OCR (optical character recognition) models, optionally with additional processing to handle some specialized types of sections (e.g., tables, forms, graphs, etc.)—as one example, information in some such specialized section types may provide information about particular attributes of an associated device or other entity, such as based on a table column and cell value, a key-value pair of a form, one or more axes and values of a graph, etc. In addition, the analysis of the text may include separating it into multiple data groupings each having an associated predicted type or topic, with non-exclusive examples including general information (e.g., names, dates, addresses, etc.), diagnosis of problems needing repair, treatments of particular repair problems, care plans for planned further repair activities to manage or solve particular repair problems, a history of past repair activities and problems, review by one repair provider of repair activities by others, etc.

FIG. 2M1-2M7 and 2N1-2N8 continue the examples of FIGS. 2A through 2L2 and 2-O through 2W, with FIG. 2M1-2M7 illustrating examples of a GUI and associated functionality that may be provided in at least some embodiments, and FIG. 2N1-2N8 illustrating an example downloaded summary of a case involving a patient entity and associated medical repair information corresponding to a damage incident (e.g., an accident)—in these illustrated examples, the GUI is used to provide information based at least in part on medical repair data (e.g., in a manner similar to that discussed with respect to FIGS. 2D through 2L2 and 2-O through 2W), but may be used to provide information and associated functionality for other types of repair data and/or associated data in other embodiments and situations.

In particular, with respect to FIG. 2M1, it includes information 200m1 that illustrates example general information including medical repair data and other information for an example legal suit involving a party who was injured, with the illustrated patient entity being John Doe, and general overview information including the patient's height and weight and BMI (body mass index) as well as overview information about a date of injury and first and last treatments-various user-selectable GUI controls are also displayed, including to enable an end user (not shown) to view a case summary, download a case summary, upload files (e.g., medical records) to be analyzed, to show notifications, to open a folder to see individual medical records to access different cases, etc. FIG. 2N1 through 2N8 include information 200n1 through 200n8, respectively, that illustrate an example of a case summary that is downloaded or otherwise provided in response to selection of the ‘Download Case Summary’user-selectable GUI control.

FIG. 2M2 through 2M7 continue the examples of FIG. 2M1, and illustrate additional screens or portions of the example GUI in respective information groups 200m2 through 200m7. The information 200m2 of FIG. 2M2 corresponds to selection of the ‘View Case Summary’ user-selectable GUI control, such as to cause display of a Timeline showing medical repair encounters organized by date/time, and multiple user-selectable tab controls to view medical repair information of various types that has been automatically extracted and categorized by the ARID system, with the tabs including ‘Medical History’, ‘Diagnoses’, ‘Prognoses’, ‘Treatments’ and ‘Hand Written’ in this example. The ‘Medical History’ tab control is selected in this example (e.g., by default upon entering the case summary view, by a prior manual selection of an end-user, etc.), with corresponding medical history documents and related information illustrated (and with additional medical history available by scrolling down, by selecting displayed information under the ‘Medical History’ tab, by selecting information shown in the ‘Timeline’, etc.)-such medical history information in this example illustrates thumbnail images of particular medical record documents and corresponding medical record data extracted from the documents about corresponding medical history information for John Doe. As previously noted, the information shown in the example GUI of FIG. 2M1-2M7 is automatically extracted at least in part from analysis of the medical record data for John Doe, as well as additional user-selectable control tabs to access further information about specific types of medical record data, such as diagnoses, treatments, etc., as well as handwritten records-the information available via the GUI may further be the result of analysis to remove duplicative records if they are present in the medical record data that is analyzed. The information 200m3 of FIG. 2M3 illustrates further details about diagnosis information, such as based on selection of the illustrated Diagnoses control tab, and which in this example illustrates thumbnail images of particular medical record documents and corresponding medical record data extracted from the documents about corresponding diagnoses for John Doe. The information 200m4 of FIG. 2M4 illustrates further details about prognosis information, such as based on selection of the illustrated Prognoses control tab, and which in this example indicates that no prognosis information was identified in the automated analysis of the medical repair document information associated with the John Doe patient entity. The information 200m5 of FIG. 2M5 illustrates further details about treatment information, such as based on selection of the illustrated Treatments control tab, and which in this example illustrates thumbnail images of particular medical record documents and corresponding medical record data extracted from the documents about corresponding medical treatments for John Doe. The information 200m6 of FIG. 2M-6 illustrates further details about handwritten record information, such as based on selection of the illustrated Hand Written records control tab, and which in this example illustrates thumbnail images of particular medical record documents and optionally corresponding medical record data extracted from handwritten information in those documents for John Doe. The information 200ml of FIG. 2M7 illustrates further details about selection of the “Imaging Report” folder of FIG. 2M1, and which in this example illustrates user-selectable links associated with particular imaging reports that are available for review. It will be appreciated that the GUI screens and associated functionality and data of FIG. 2M1-2M7 and 2N1-2N8 are provided for purposes of example, and that the invention is not limited to the details shown.

Thus, as discussed in greater detail elsewhere herein, the types of repair information discussed with respect to FIGS. 2A-2W may be used in various manners, including to be used to initiate further repair activities (e.g., in an automated manner), to be displayed to users, used to provide responses to queries, etc. It will be also be appreciated that questions and other queries in a medical domain may be of various types, such as related to a particular diseases or other medical conditions, particular medical symptoms, particular medicines and other treatments and other health-related activities involved in preventative actions or other actions to improve fitness and wellbeing, particular surgeries and other medical procedures, particular medical devices and other health-related objects used with respect to medical treatments and other health-related activities, particular patients or groups of patients, etc., and that the summarization and encoding of information for a medical domain may include identifying and encoding information about some or all such elements (e.g., diseases or other conditions, symptoms, treatments, medical procedures, medical devices and other health-related objects, patients, etc.). In addition, it will be appreciated that a variety of other types of information may be available and used in other embodiments, including to provide a response in a different format than is shown in the examples of FIGS. 2A-2W.

Various details have been provided with respect to FIGS. 2A-2W, but it will be appreciated that the provided details are non-exclusive examples included for illustrative purposes, and other embodiments may be performed in other manners without some or all such details.

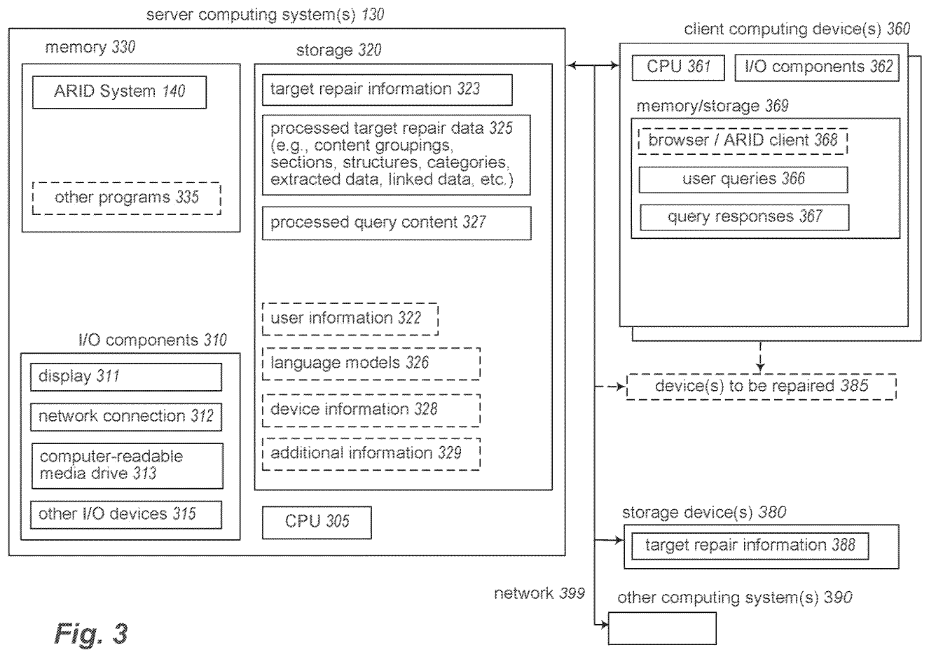

FIG. 3 is a block diagram illustrating an embodiment of one or more server computing systems 130 executing an implementation of an ARID system 140—the server computing system(s) and ARID system may be implemented using a plurality of hardware components that form electronic circuits suitable for and configured to, when in combined operation, perform at least some of the techniques described herein. In the illustrated embodiment, each server computing system 130 includes one or more hardware central processing units (“CPU”) or other hardware processors 305, various input/output (“1/0”) components 310, storage 320, and memory 330, with the illustrated 1/0 components including a display 311, a network connection 312, a computer-readable media drive 313, and other 1/0 devices 315 (e.g., keyboards, mice or other pointing devices, microphones, speakers, GPS receivers, etc.).

The server computing system(s) 130 and executing ARID system 140 may communicate with other computing systems and devices via one or more networks 399 (e.g., the Internet, one or more cellular telephone networks, etc.), such as user client computing devices 360 (e.g., used to supply queries; receive responses; and use the received response information, such as to implement automated repairs to associated devices 385 and/or to display or otherwise present response information to users of the client computing devices), optionally one or more devices 385 to be repaired (e.g., if the devices include networking capabilities or other data transmission capabilities), optionally other storage devices 380 (e.g., used to store and provide information 388 for one or more target domains/repair areas), and optionally other computing systems 390.

In the illustrated embodiment, an embodiment of the ARID system 140 executes in memory 330 in order to perform at least some of the described techniques, such as by using the processor(s) 305 to execute software instructions of the system 140 in a manner that configures the processor(s) 305 and computing system 130 to perform automated operations that implement those described techniques. The illustrated embodiment of the ARID system may include one or more components and/or modules, not shown, to each perform portions of the functionality of the ARID system, and the memory may further optionally execute one or more other programs 335. The ARID system 140 may further, during its operation, store and/or retrieve various types of data on storage 320 (e.g., in one or more databases or other data structures), such as various types of user information 322, target repair information 323 (e.g., local copies of some or all of information 388 on remote systems such as storage devices 380; domain-specific information to use in customizing the analyzing of content for a domain and/or other additional supplemental information; etc.), processed target repair data 325 of one or more types (e.g., content groupings; encounters; sections; structural elements; associated category and/or topic information; extracted data, optionally associated together into data groupings based on the documents/content groupings/encounters/sections/etc. from which it is extracted; sets of linked data, such as associated with particular entities; etc.), processed query-based content 327 (e.g., candidate data groupings, generated responses, etc.), optionally language models 326 to use in analyzing content for particular repair domains, optionally device-specific information 328 (e.g., related to devices to be repaired) or information specific to other entities, and/or various other types of optional additional information 329.

Some or all of the user client computing devices 360 (e.g., mobile devices), devices 385 to be repaired, storage devices 380, and other computing systems 390 may similarly include some or all of the same types of components illustrated for server computing system 130. As one non-limiting example, the computing devices 360 are each shown to include one or more hardware CPU(s) 361, 1/0 components 362, and memory and/or storage 369, with a browser and/or ARID system client program 368 optionally executing in memory to interact with the ARID system 140 and present or otherwise use query responses 367 that are received from the ARID system for submitted user queries 366. While particular components are not illustrated for the other devices/systems 380 and 385 and 390, it will be appreciated that they may include similar and/or additional components.

It will also be appreciated that computing system 130 and the other systems and devices included within FIG. 3 are merely illustrative and are not intended to limit the scope of the present invention. The systems and/or devices may instead each include multiple interacting computing systems or devices, and may be connected to other devices that are not specifically illustrated, including via Bluetooth communication or other direct communication, through one or more networks such as the Internet, via the Web, or via one or more private networks (e.g., mobile communication networks, etc.). More generally, a device or other computing system may comprise any combination of hardware that may interact and perform the described types of functionality, optionally when programmed or otherwise configured with particular software instructions and/or data structures, including without limitation desktop or other computers (e.g., tablets, slates, etc.), database servers, network storage devices and other network devices, smart phones and other cell phones, consumer electronics, wearable devices, digital music player devices, handheld gaming devices, PDAs, wireless phones, Internet appliances, and various other consumer products that include appropriate communication capabilities. In addition, the functionality provided by the illustrated ARID system 140 may in some embodiments be distributed in various components, some of the described functionality of the ARID system 140 may not be provided, and/or other additional functionality may be provided.

It will also be appreciated that, while various items are illustrated as being stored in memory or on storage while being used, these items or portions of them may be transferred between memory and other storage devices for purposes of memory management and data integrity. Alternatively, in other embodiments some or all of the software components and/or modules and/or systems may execute in memory on another device and communicate with the illustrated computing systems via inter-computer communication. In some embodiments, some or all of the described techniques may be performed by hardware means that include one or more processors and/or memory and/or storage when configured by one or more software programs (e.g., by some or all of the ARID system 140 executing on server computing systems 130) and/or data structures, such as by execution of software instructions of the one or more software programs and/or by storage of such software instructions and/or data structures, and such as to perform algorithms as described in the flow charts and other disclosure herein. Furthermore, in some embodiments, some or all of the systems and/or components may be implemented or provided in other manners, such as by consisting of one or more means that are implemented partially or fully in firmware and/or hardware (e.g., rather than as a means implemented in whole or in part by software instructions that configure a particular CPU or other processor), including, but not limited to, one or more application-specific integrated circuits (ASICs), standard integrated circuits, controllers (e.g., by executing appropriate instructions, and including microcontrollers and/or embedded controllers), field-programmable gate arrays (FPGAs), complex programmable logic devices (CPLDs), etc. Some or all of the components, systems and data structures may also be stored (e.g., as software instructions or structured data) on a non-transitory computer-readable storage mediums, such as a hard disk or flash drive or other non-volatile storage device, volatile or non-volatile memory (e.g., RAM or flash RAM), a network storage device, or a portable media article (e.g., a DVD disk, a CD disk, an optical disk, a flash memory device, etc.) to be read by an appropriate drive or via an appropriate connection. The systems, components and data structures may also in some embodiments be transmitted via generated data signals (e.g., as part of a carrier wave or other analog or digital propagated signal) on a variety of computer-readable transmission mediums, including wireless-based and wired/cable-based mediums, and may take a variety of forms (e.g., as part of a single or multiplexed analog signal, or as multiple discrete digital packets or frames). Such computer program products may also take other forms in other embodiments. Accordingly, embodiments of the present disclosure may be practiced with other computer system configurations.

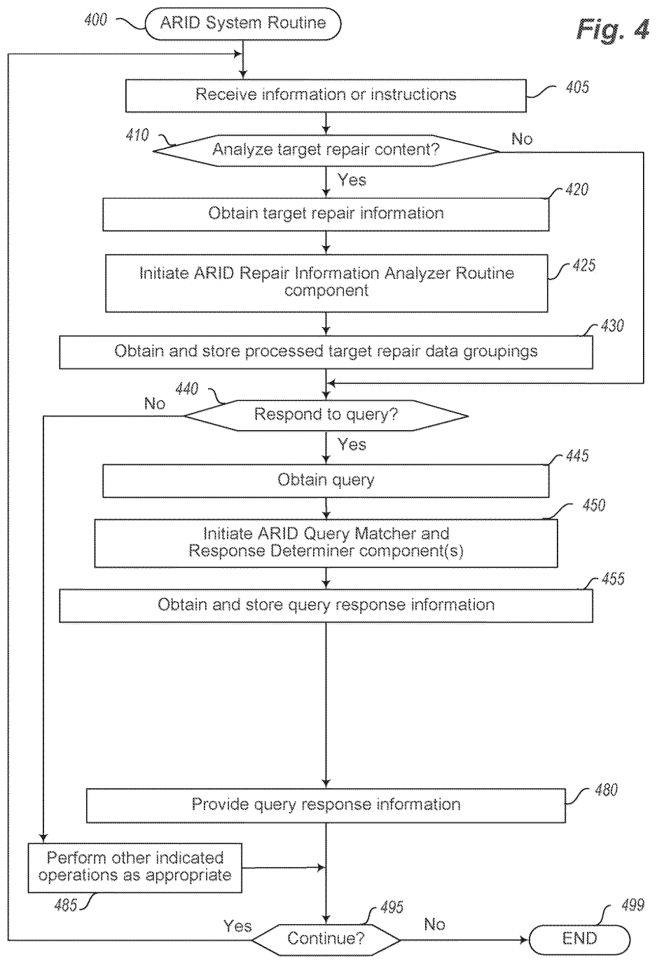

FIG. 4 is a flow diagram of an example embodiment of an ARID system routine 400. The routine may be provided by, for example, execution of the ARID system 140 of FIGS. 1A and 1B, and/or the ARID system 140 of FIG. 3, and/or corresponding functionality discussed with respect to FIGS. 2A-2W and elsewhere herein, such as to analyze repair information and determine resulting repair data of one or more types, and to use that repair data in one or more manners, including to automatically determine responses to received queries (e.g., with respect to computing devices or other devices, with respect to medical information, etc.). In the illustrated embodiment, the routine analyzes information about one or more domains of interest at various times, such as to pre-process information about a particular domain (e.g., as instructed by a human operator of the ARID system, as requested by a third-party entity, etc.) to determine at least some types of domain-specific information for use in responding to later queries based on such information, while in other embodiments the routine may instead dynamically generate some or all types of domain-specific information in response to requests from users or other entities in at least some situations.

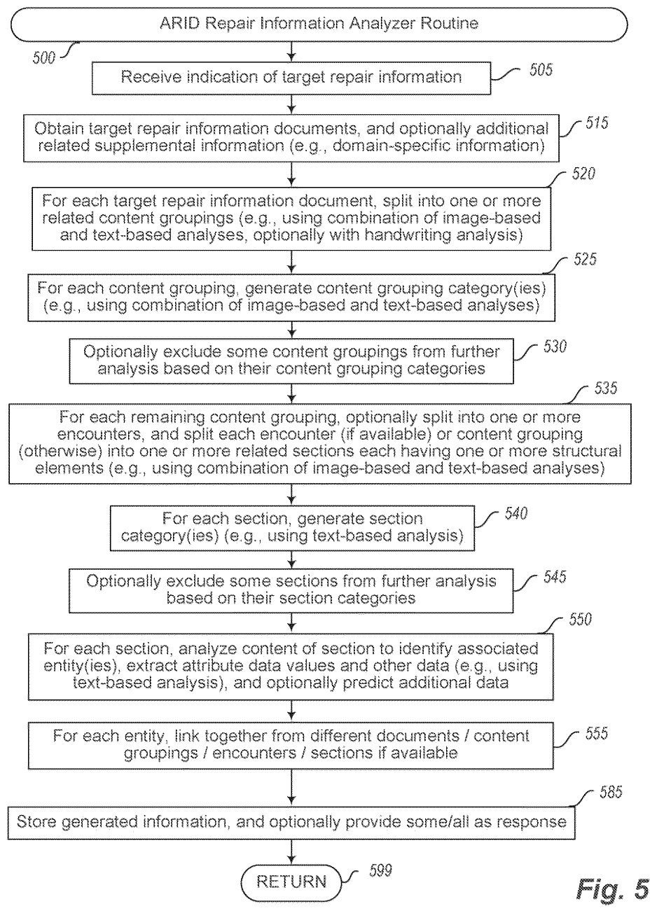

In the illustrated embodiment, the routine 400 begins at block 405, where instructions or other information is received. The routine continues to block 410, where it determines if the instructions or other information received in block 405 are to analyze target repair content, such as for a target repair area or other target domain, and if so continues to block 420 where it retrieves or otherwise obtains the target information (e.g., uses information received in block 405) to be analyzed (e.g., multiple documents that are part of information for the target domain). In block 425, the routine then initiates execution of an ARID Target Repair Information Analyzer component routine to analyze and summarize information from the target domain (along with an indication of that target domain), such as corresponding to component 166 of FIGS. 1A and 1B, and with one example of a corresponding routine for performing such activities discussed further in FIG. 5. In block 430, the routine then receives the processed target repair data groupings from block 425, and stores the information for subsequent use. It will be appreciated that, while the routine indicates proceeding to block 430 immediately after block 425, in other embodiments the routine may operate in an asynchronous manner such that other operations are performed (e.g., corresponding to handling another set of instructions or information that are received in block 405, such as from a different user and/or for a different entity) while waiting for a response from block 425, and that the operations of block 425 may be performed in a substantially immediate manner (e.g., less than one second, less than 10 seconds, less than one minute, etc.) in at least some embodiments.

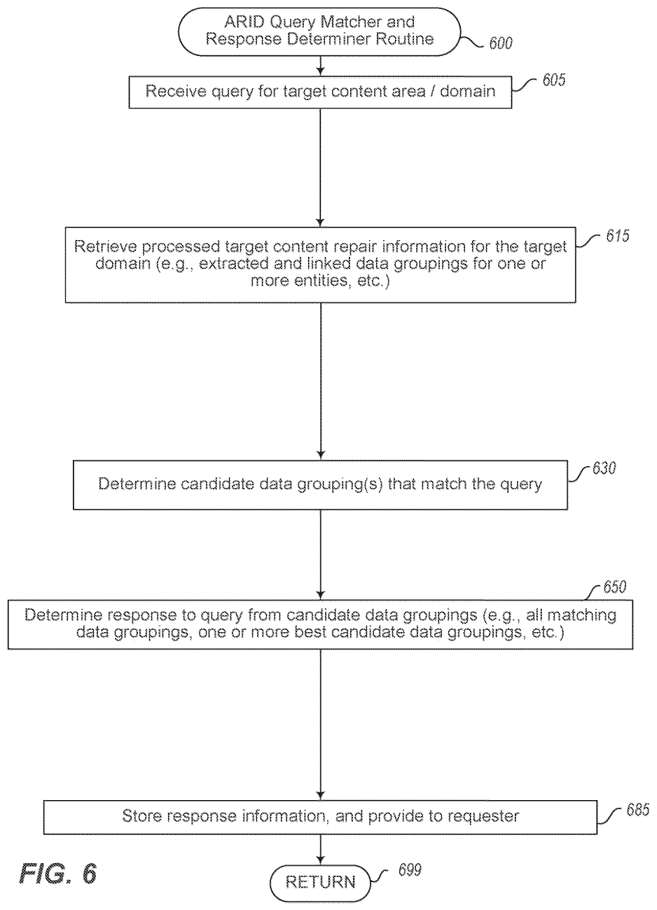

After block 430, or if it is instead determined in block 410 that the information or instructions received in block 405 are not to analyze content for a target repair area or other target domain, the routine continues to block 440, where it determines if the information or instructions received in block 405 are to respond to a received query, and if not continues to block 485. Otherwise, the routine continues to block 445 where it obtains a query (e.g., in natural language form, using information received in block 405, etc.), and then proceeds to block 450 to initiate execution of ARID Query Matcher and Response Determiner components' routines, such as to correspond to components 167 and 169 of FIGS. 1A and 1B, and with one example of a corresponding routine for performing such activities discussed further in FIG. 6. In block 455, the routine then receives query response information from block 450, and stores the information for later use, with the query response information then provided in block 480 as a response to the received query. It will be appreciated that, while the routine indicates proceeding to block 455 immediately after block 450, in other embodiments the routine may operate in an asynchronous manner such that other operations are performed (e.g., corresponding to handling another set of instructions or information that are received in block 405, such as from a different user or other entity) while waiting for a response from block 450, and that the operations of block 450 may be performed in a substantially immediate manner (e.g., in a real-time or near-real-time manner, such as a fraction of a second, a second, less than 10 seconds, etc.) in at least some embodiments.

In block 485, the routine proceeds to perform one or more other indicated operations as appropriate, with non-exclusive examples of such other operations including retrieving and providing previously determined or generated information (e.g., previous user queries, previously determined responses to user queries, previously summarized repair data for one or more target domains, etc.), receiving and storing information for later use (e.g., information about one or more target domains, such as documents to be analyzed, domain-specific language models, etc.), providing information about how one or more previous query responses were determined, performing housekeeping operations, etc.

After blocks 480 or 485, the routine continues to block 495 to determine whether to continue, such as until an explicit indication to terminate is received, or alternatively only if an explicit indication to continue is received. If it is determined to continue, the routine returns to block 405 to await further information or instructions, and if not continues to block 499 and ends.

FIG. 5 is a flow diagram of an example embodiment of an ARID Repair Information Analyzer routine 500. The routine may be provided by, for example, execution of the ARID Repair Information Analyzer component 166 of FIGS. 1A-1B and/or a corresponding component (not shown) of the ARID system 140 of FIG. 3 and/or with respect to corresponding functionality discussed with respect to FIGS. 2A-2W and elsewhere herein, such as to analyze and summarize repair information (e.g., for a particular entity, for a particular target domain, etc.). In addition, in at least some situations, the routine 500 may be performed based on execution of block 425 of FIG. 4, with resulting information provided and execution control returning to that location when the routine 500 ends. In this example, the routine 500 is performed with respect to an initial analysis of a particular group of repair data (e.g., medical records and optionally other associated health-related information for one or more patient entities), but in other embodiments may be similarly performed to update previously analyzed information, such as to analyze additional documents that become available for an entity and/or domain after a prior analysis of other documents and use of resulting information has been completed. Furthermore, in a manner similar to that of the other routines, the routine 500 may determine repair data in advance of the use of that determined repair data, and/or dynamically in response to a request for such information.

The illustrated embodiment of the routine 500 begins at block 505, where an indication of target repair information to be analyzed (e.g., particular documents with some or all of the target repair information) is received. In block 515, the routine then obtains documents with the target repair information and optionally additional domain-specific information (e.g., domain-specific language models, etc.), such as by using currently provided repair data (e.g., received in block 505), using previously stored repair information, by searching for or otherwise dynamically identifying corresponding repair information, etc. In block 520, the routine then, for each target repair information documents, split the content of the document into one or more related content groupings, such as by using a combination of image-based and text-based analyses as discussed in greater detail elsewhere herein. In block 525, the routine then, for each content grouping, generates one or more associated content grouping categories, such as by using text-based analyses (optionally in combination with image-based analyses) as discussed in greater detail elsewhere herein. In block 530, the routine then optionally excludes some content groupings from further analysis, such as based on their content grouping categories. In block 535, the routine then, for each remaining content grouping, optionally splits the content of that content grouping into one or more encounters, and then splits each set of content for an encounter (if available) or for the content grouping (if not) and to one or more related sections each having one or more structural elements, such as by using a combination of image-based and text-based analyses as discussed in greater detail elsewhere herein. After block 535, the routine in block 540 then, for each section, generates one or more section categories, such as by using a text-based analysis as discussed in greater detail elsewhere herein. In block 545, the routine then optionally excludes some sections from further analysis based at least in part on their section categories, in block 550, for each remaining section, analyzes the content of that section to identify associated entities, to extract attribute data values and other repair data (e.g., by using a text-based analysis), including to optionally predict additional data. In block 555, for each entity, prepared data is linked together from different documents and/or content groupings and/or encounters and/or sections if available, including to optionally add the currently determined repair data to previously stored repair data for that entity.

After block 555, the routine continues to block 585 to store the generated information for later use (e.g., in tables of one or more SOL databases), and to optionally provide some or all of the generated information to the requester that initiated invocation of the routine 500. After block 585, the routine continues to block 599 and ends.

FIG. 6 is a flow diagram of an example embodiment of an ARID Query Matcher and Response Determiner routine 600. The routine may be provided by, for example, execution of the ARID Query Matcher component 167 and ARID Response Determiner component 169 of FIGS. 1A-1B and/or of one or more corresponding components (not shown) of the ARID system 140 of FIG. 3 and/or with respect to corresponding functionality discussed with respect to FIGS. 2A-2W and elsewhere herein (e.g., to implement some or all of the example GUI functionality of FIG. 2M1-2M7), such as to respond to a received query by determining a response to the query using corresponding summarized repair data. The routine 600 may be initiated by, for example, execution of block 450 of FIG. 4, with resulting information provided and execution control returning to that location when the routine 600 ends. In addition, in a manner similar to that of the other routines, the routine 600 may dynamically determine and provide responses to received queries in the illustrated embodiment, but in other embodiments may determine and store responses to some or all queries in advance of receiving corresponding new user queries (e.g., based on instructions received from a human operator of the ARID system, based on previous queries by other users, etc.) and use such stored responses when such new queries are received. In addition, in the illustrated embodiment, the determination of encoded domain information in FIG. 5 is illustrated as being performed separately from the use of such information in FIG. 6, although in other embodiments such activities may be performed in other manners, including to dynamically perform some or all of the activities of FIGS. 5 and 6 in response to a received user query or other corresponding instruction.

The illustrated embodiment of the routine 600 begins in block 605, where a query is received corresponding to repair data of one or more types (e.g., for a particular associated entity). In block 615, the routine then retrieves corresponding processed target repair data (e.g., sets of linked data groupings, etc.), such as by performing one or more corresponding retrieval actions for stored data (e.g., SOL queries on one or more SOL databases), although in other embodiments may instead dynamically generate such information (e.g., if the user query corresponds to a new type for which previously stored information is not available, if updates to underlying information of the requested type are available but not yet analyzed, etc.). In block 630, the routine then determines a subset of the candidate data groupings that match the query(ies). In block 650, the routine then determines a response to the query from the candidate data groupings, such as all of the candidate data groupings, by analyzing the candidate data groupings to select one or more of the candidate data groupings (or subsets of one or some or all of the candidate data groupings) that are determined to best match the query, etc. After block 660, the routine continues to block 685 to store the determined response information for later use, and to provide that determined information to the requester that initiated invocation of the routine 600. After block 685, the routine continues to block 699 and ends.

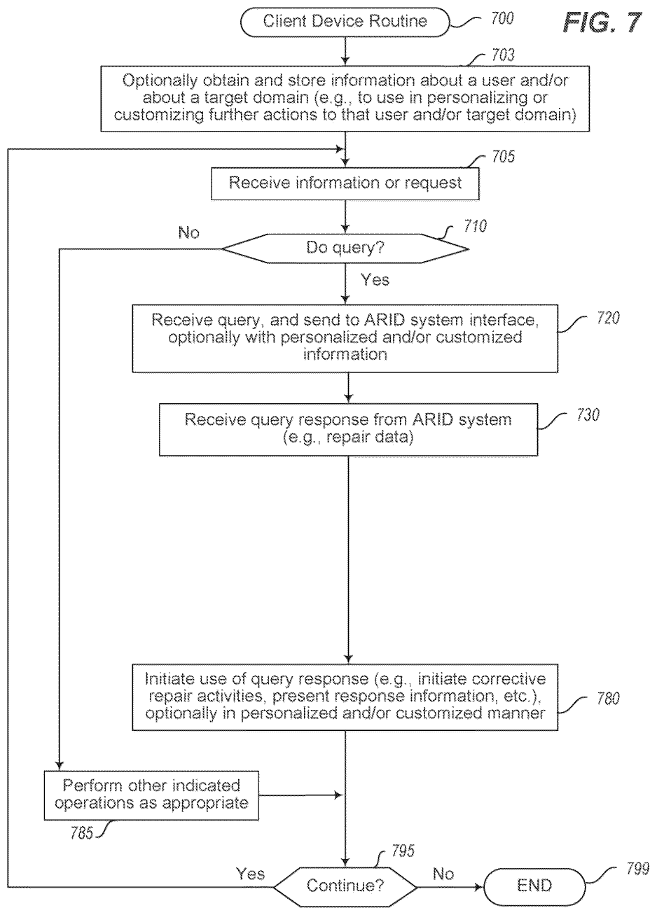

FIG. 7 is a flow diagram of an example embodiment of a client device routine 700. The routine may be provided by, for example, operations of a client computing device 110 of FIGS. 1A-1B and/or a client computing device 360 of FIG. 3 and/or with respect to corresponding functionality discussed with respect to FIGS. 2A-2W and elsewhere herein, such as to interact with users or other entities who submit queries (or other information) to the ARID system, to receive responses (or other information) from the ARID system, and to use the received information in one or more manners (e.g., to automatically implement corrective repair activities in accordance with a received response that includes corresponding executable instructions; to otherwise assist in initiating corrective or preventative repair activities in accordance with a received response that includes corresponding information, such as by displaying or otherwise presenting at least some such information to one or more users to enable them to perform at least some of the corresponding activities; to otherwise assist in providing information from the determined response to one or more users or other requesting entities; etc.).

The illustrated embodiment of the routine 700 begins at block 703, where information is optionally obtained and stored about the user of the client device and/or about a target domain, such as for later use in personalizing or otherwise customizing further actions to that user and/or that target domain. The routine then continues to block 705, where information or a request is received. In block 710, the routine determines if the information or request received in block 705 is to perform a query, and if not continues to block 785. Otherwise, the routine continues to block 720, where it receives the query (e.g., in a natural language format, such as free form text), and sends a query to the ARID system interface to obtain a corresponding response, optionally after personalizing and/or customizing the information to be provided to the ARID system (e.g., to add information specific to the user, such as location, demographic information, preference information, etc.; to add an indication of one or more specific target domains to use; to add an indication of a specific target entity for which to gather repair data; etc.). In block 730, the routine then receives a response to the query from the ARID system, such as to include repair data. In block 780, the routine then initiates use of the received query response information, such as to initiate automated repair activities, to display or otherwise present response information to the user, etc., including to optionally perform such use in a personalized and/or customized manner (e.g., to perform a display or other presentation in accordance with preference information for the user, to select a type of action to take based on information specific to the user, etc.). It will be appreciated that, while the routine indicates proceeding to block 730 immediately after block 725, in other embodiments the routine may operate in an asynchronous manner such that other operations are performed (e.g., corresponding to handling another set of instructions or information that are received in block 705, such as from a different user or other entity) while waiting for a response from block 725, and that the operations of block 725 may be performed in a substantially immediate manner (e.g., less than one second, less than 10 seconds, less than one minute, etc.) in at least some embodiments.

In block 785, the routine instead performs one or more other indicated operations as appropriate, with non-exclusive examples including sending information to the ARID system of other types (e.g., information about a new target domain and/or entity for which to analyze and summarize information before corresponding user queries are received, information to be processed for an indicated target domain and/or entity, etc.), receiving and responding to requests for information about previous user queries and/or corresponding responses for a current user and/or client device, receiving and store information for later use in personalization and/or customization activities, receiving and responding to indications of one or more housekeeping activities to perform, etc.

After blocks 780 or 785, the routine continues to block 795 to determine whether to continue, such as until an explicit indication to terminate is received (or alternatively only if an explicit indication to continue is received). If it is determined to continue, the routine returns to block 705, and if not continues to block 799 and ends.

It will be appreciated that in some embodiments the functionality provided by the routines discussed above may be provided in alternative ways, such as being split among more routines or consolidated into fewer routines. Similarly, in some embodiments illustrated routines may provide more or less functionality than is described, such as when other illustrated routines instead lack or include such functionality respectively, or when the amount of functionality that is provided is altered. In addition, while various operations may be illustrated as being performed in a particular manner (e.g., in serial or in parallel) and/or in a particular order, those skilled in the art will appreciate that in other embodiments the operations may be performed in other orders and in other manners. Those skilled in the art will also appreciate that the data structures discussed above may be structured in different manners, such as by having a single data structure split into multiple data structures or by having multiple data structures consolidated into a single data structure. Similarly, in some embodiments illustrated data structures may store more or less information than is described, such as when other illustrated data structures instead lack or include such information respectively, or when the amount or types of information that is stored is altered.

From the foregoing it will be appreciated that, although specific embodiments have been described herein for purposes of illustration, various modifications may be made without deviating from the spirit and scope of the invention. Accordingly, the invention is not limited except as by the claims that are specified and the elements recited therein. In addition, while certain aspects of the invention may be presented at times in certain claim forms, the inventors contemplate the various aspects of the invention in any available claim form. For example, while only some aspects of the invention may be recited at a particular time as being embodied in a computer-readable medium, other aspects may likewise be so embodied.

What is claimed is:

1. A computer-implemented method comprising:
obtaining, by one or more computing systems, a plurality of documents with document contents describing a plurality of activities performed for an indicated entity by multiple service providers at a plurality of times, the contents including data of multiple types about the indicated entity using both structured and unstructured data formats and having information about values for attributes of the indicated entity;
determining, by the one or more computing systems and using a combination of image-based analysis and text-based analysis of the document contents, the data of the multiple types about the indicated entity, including:
determining, by the one or more computing systems, a plurality of content groupings within the plurality of documents, including, for each of at least one document of the plurality of documents, determining multiple content groupings within the document that were generated separately from each other before being combined in the document, including:
analyzing images of pages of the document to identify one or more candidate content grouping boundaries between content groupings by determining whether images of subsequent pages of the document are part of a same content grouping or multiple different content groupings; and
analyzing surrounding text from contents of the document around each of at least some of the candidate content grouping boundaries to verify that the surrounding text belongs to multiple different content groupings;
determining, by the one or more computing systems, a plurality of sections within at least some of the content groupings, including identifying structural elements of multiple types within a content grouping based at least in part on analysis of one or more images showing the content grouping, and combining, automatically by the one or more computing systems and without user input, some of the structural elements to form one or more sections of the plurality of sections, and selecting other of the structural elements to each be one or more other sections of the plurality of sections;
extracting, by the one or more computing systems, at least some of the data of the multiple types about the indicated entity from at least some of the determined sections, including extracting data from both structured and unstructured data formats and using the extracted data to determine the values for the attributes of the indicated entity;
linking, by the one or more computing systems and for each of at least some of the multiple types of data, extracted data of that type from multiple documents of the plurality of documents, to form an aggregated summary of data for the indicated entity for each of the at least some types;
presenting, by the one or more computing systems, a graphical user interface (GUI) to a user that includes the aggregated summary of data for the indicated entity;
receiving, by the one or more computing systems and via one or more interactions by the user with the GUI, a query related to a subset of the aggregated summary of data for the indicated entity;
determining, by the one or more computing systems and in response to the received query, response information with extracted data for the indicated entity that is of at least one of the multiple types and that includes at least one of the values for the attributes of the indicated entity; and
providing, by the one or more computing systems, the determined response information to the received query.

2. The method of claim 1, wherein the determining of the plurality of content groupings includes, for at least one candidate content grouping boundary, determining whether a portion on a subsequent page is a continuation of content on a preceding page.

3. The method of claim 1, wherein the determining of the plurality of sections includes using each of one or more of the structural elements as a separate section of the plurality of sections.

4. The method of claim 1, wherein the determining of the plurality of sections includes combining two or more of the structural elements to form a single section of the plurality of sections.

5. The method of claim 1, wherein the structural elements include structural elements of different types selected from the group consisting of lines, text blocks, tables, checkboxes, handwritten entries, printed labels, and image regions.

6. The method of claim 1, wherein the extracting includes using a natural language processing model trained for analyzing information to analyze text of at least some of the plurality of sections and to identify one or more data groupings each having some of the data.

7. The method of claim 6, wherein the extracting includes determining, for each of at least some identified data groupings, one of the multiple types of data for the identified data grouping.

8. The method of claim 1, further comprising determining, by the one or more computing systems, a timeline that is associated with at least some extracted data and that corresponds to the plurality of times.

9. The method of claim 8, wherein the response information includes at least some of the timeline.

10. A non-transitory computer-readable medium having stored contents that, when executed by one or more computing systems, cause the one or more computing systems to perform automated operations comprising:
obtaining a plurality of documents having document contents that describe activities associated with an indicated entity, the document contents including data of multiple types using both structured and unstructured data formats;
determining a plurality of content groupings within the plurality of documents, including, for each of at least one document of the plurality of documents, determining multiple content groupings within the document that were generated separately from each other before being combined in the document, including:
analyzing images of pages of the document to identify one or more candidate content grouping boundaries between content groupings by determining whether images of subsequent pages of the document are part of a same content grouping or multiple different content groupings; and
analyzing surrounding text from contents of the document around each of at least some of the candidate content grouping boundaries to verify that the surrounding text belongs to multiple different content groupings;
determining a plurality of sections within at least some of the content groupings, including identifying structural elements of multiple types within a content grouping based at least in part on analysis of one or more images showing the content grouping, and combining, automatically and without user input, some of the structural elements to form one or more sections of the plurality of sections, and selecting other of the structural elements to each be one or more other sections of the plurality of sections;
extracting at least some of the data of the multiple types from at least some of the plurality of sections; and
linking extracted data of a same type from multiple documents of the plurality of documents to form an aggregated summary for the indicated entity.

11. The non-transitory computer-readable medium of claim 10, wherein the automated operations further comprise determining, for each of at least some of the plurality of content groupings, one of multiple content grouping categories for the content grouping.

12. The non-transitory computer-readable medium of claim 11, wherein the automated operations further comprise determining a content grouping subset for further analysis by excluding at least one content grouping based at least in part on the determined content grouping category for the excluded content grouping.

13. The non-transitory computer-readable medium of claim 10, wherein the automated operations further comprise determining, for each of at least some of the plurality of sections, one of multiple section categories for the section.

14. The non-transitory computer-readable medium of claim 13, wherein the automated operations further comprise determining a section subset for further analysis by excluding at least one section based at least in part on the determined section category for the excluded section.

15. The non-transitory computer-readable medium of claim 10, wherein the extracting includes applying a natural language processing model to text of at least some of the plurality of sections to identify data groupings and corresponding data types.

16. The non-transitory computer-readable medium of claim 10, wherein the indicated entity is a device and the extracted data includes repair data associated with the device.

17. The non-transitory computer-readable medium of claim 10, wherein the indicated entity is a patient and the extracted data includes medical data associated with the patient.

18. The non-transitory computer-readable medium of claim 10, wherein the automated operations further comprise generating a timeline associated with at least some extracted data and corresponding to multiple times associated with the plurality of documents.

19. The non-transitory computer-readable medium of claim 10, wherein the automated operations further comprise presenting a graphical user interface that includes at least some of the aggregated summary and receiving, via one or more interactions with the graphical user interface, a query related to a subset of the aggregated summary.

20. A non-transitory computer-readable medium having stored contents that cause one or more computing systems to perform automated operations, the automated operations including at least:
determining, by the one or more computing systems, medical data of multiple types for a patient by performing a combination of image-based and text-based analyses of a plurality of content groupings that are associated with a plurality of medical service providers and that have contents including at least the medical data and that each represents at least one interaction session of one of the medical service providers with the patient, including:
determining, by the one or more computing systems, the plurality of sections within at least some content groupings of the plurality of content groupings, including, for each of one or more of the at least some content groupings, determining multiple sections within the content grouping that each includes content in a format different than surrounding sections within the content grouping, including:
identifying, by the one or more computing systems, structural elements of multiple types within the content grouping based at least in part on analysis of one or more images showing the content grouping, and
combining, automatically by the one or more computing systems and without user input, some of the structural elements to form one or more sections of the multiple sections, and selecting other of the structural elements to each be one or more other sections of the multiple sections;
extracting, by the one or more computing systems, the medical data of the multiple types for the patient from at least some of the plurality of sections; and
linking, by the one or more computing systems and for each of at least some of the multiple types of medical data, extracted medical data of that type from multiple content groupings associated with multiple medical service providers, to form an aggregated summary of medical data for the patient for each of the at least some types; and
providing, by the one or more computing systems, at least some of the determined medical data of the multiple types for the patient, to enable further use of the provided at least some determined medical data, including:
presenting, by the one or more computing systems, a graphical user interface (GUI) to a user that includes at least some of the extracted medical data of the multiple types for the patient; and
receiving, by the one or more computing systems and via one or more interactions by the user with the GUI, a query related to a subset of the extracted medical data of the multiple types for the patient,
and wherein the providing of the at least some of the determined medical data of multiple types is performed in response to the received query and includes providing the subset of the extracted medical data of the multiple types for the patient.
